(19) United States
(12) Patent Application Publication
(10) Pub. No.: US 2010/0004316 A1
(43) Pub. Date: Jan. 7, 2010

(54) MULTIFUNCTIONAL CARRIERS FOR THE DELIVERY OF NUCLEIC ACIDS AND METHODS OF USE THEREOF

(75) Inventors: Zheng-Rong Lu, Salt Lake City, UT (US); Xu-Li Wang, Salt Lake City, UT (US)

Correspondence Address: GARDNER GROFF GREENWALD & VILLANUEVA. PC, 2018 POWERS FERRY ROAD, SUITE 800, ATLANTA, GA, 30339, US

(73) Assignee: UNIVERSITY OF UTAH RESEARCH FOUNDATION, Salt Lake City, UT (US)

(21) Appl. No.: 12/443,002

(22) PCT Filed: Sep. 27, 2007
(86) PCT No.: PCT/US2007/079651

§ 371 Date: Apr. 21, 2009

Related U.S. Application Data
(60) Provisional application No. 60/827,440, filed on Sep. 29, 2006.

(51) Int. Cl. A61K 48/00; A61K 31/713; C07K 14/00; C12N 5/00

(52) U.S. Cl. 514/44 A; 530/409; 435/375

(57) ABSTRACT

Described herein are multifunctional compounds useful as devices for the delivery of nucleic acids to cells. Also described herein are methods for using the multifunctional compounds.

### CROSS REFERENCE TO RELATED APPLICATIONS

This application claims priority upon U.S. provisional application Ser. No. 60/827,440, filed Sep. 29, 2006. This application is hereby incorporated by reference in its entirety for all of its teachings.

### ACKNOWLEDGEMENT

This invention was made with government support under grant EB000489 awarded by the National Institute of Biomedical Imaging and Bioengineering of the National Institutes of Health. The Government has certain rights to this invention.

### BACKGROUND

Gene therapy has emerged as an exiting new approach for the treatment of human diseases by transferring genetic materials into human cells to express or regulate the gene for therapeutic purposes. The development of safe and efficient gene vectors or vehicles remains a central challenge to the success of nucleic-acid based therapies.

The delivery systems for nucleic acids can be categorized as viral delivery systems and nonviral delivery systems. Viral vectors are based on attenuated viruses, such as adenoviruses, adeno-associated viruses, herpesviruses, lentiviruses and retroviruses. Viral vectors have high efficiency in terms of delivering and expressing genes in human cells and are popular vectors in research and development. However, clinical application of viral vectors is seriously overshadowed by their potential toxicity, particularly after the recent incidents of leukemia development in X-SCID patients and patient death in clinical trials with viral vectors. The toxicity of viral vectors is associated with impurity of the viral vectors and the host immune response to the viral vectors. Moreover, viral vectors are not suitable for multiple treatments because the host immune system will develop antibodies against the vectors after one or two administrations.

Nonviral delivery systems of nucleic acids include all the systems other than viral systems. Various nonviral approaches, including direct injection, hydrodynamic delivery, chemical modification, peptides, liposomes, and cationic polymers, have been used for the delivery of nucleic acids such as, for example, siRNAs. As compared to viral delivery systems, these nonviral systems are easy to use and can be readily produced in a large scale. Most importantly, they are not immunogenic and are less likely to stimulate host immune response. The main drawback for currently available nonviral delivery systems is the low delivery efficiency of nucleic acids into target cells. In spite of their low delivery efficiency, nonviral delivery systems have become increasingly preferable in terms of safety, non-immunogenicity, ease of production, and low cost.

Thus, what is needed is a delivery carrier that is non-toxic, non-immunogenic, and protects the nucleic acid from enzymatic degradation during the delivery process. It is also desirable that the delivery system prevents rapid elimination from the body, facilitate specific uptake by target tissue and target cells, and rapidly release the nucleic acid intracellularly at the site of action. Described herein are multifunctional compounds useful as carriers for the delivery of nucleic acids to cells. The multifunctional delivery systems described herein address many of the shortcomings of current delivery systems.

### SUMMARY

Described herein are multifunctional carriers (MFC) useful for the delivery of nucleic acids to cells. Also described herein are methods for using the delivery systems. The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows the synthetic scheme for preparing a monomeric multifunctional carrier THCO.

FIG. 2 shows the structures of several MFCs produced herein.

FIG. 3 shows THCO retarded DNA migration in gel at N/P ratio of 1 and above.

FIG. 4 shows the time dependent oxidation profile of THCO by Ellman's reagent THCO and THCO/DNA complexes.

FIG. 5 shows the auto-oxidation profile of THCO and THCO/siRNA complexes (N/P=10).

FIG. 6 shows the competition of thiol consumption of THCO between auto-oxidation and reactivity with maleimide.

FIG. 7 shows the relative cell viability (%) vs. concentration of THCO and PEI.

FIG. 8 shows the cytotoxicity of THCO in U87 cells comparing with DOTAP and PEI.

FIG. 9 shows the cellular uptake of fluorescence-tagged THCO/siRNA complexes with or without pegylation in U87 cells. Pegylation of the nanoparticles reduced non-specific cell uptake.

FIG. 10 shows the transfection efficiency of THCO when compared to other commercially available transfection agents.

FIG. 11 shows the fluorescence image of GFP-transfected MBA-231 cells using the following transfection agents: A) THCO; B) Transfast; C) DOTAP.

FIG. 12 shows THCO/siRNA (N/P=8) mediated luciferase gene silencing in U87-Luc cells (siRNA concentration was fixed at 20 nM in all cases; Transfast™ and DOTAP were used as controls). Anti-Luc siRNA or non-specific siRNA was complexed with A: Unmodified THCO; B: 0.5% PEGylated THCO; C: 2.5% PEGylated THCO; D: Transfast; or E: DOTAP and the resulted siRNA complexes were incubated with U87-Luc cells for 4 h followed by replacement of growth medium. Luciferase activity was assessed 44 h post-transfection. Transfection experiments were performed triplicate in either serum-free medium (a) or 10% FBS medium (b).

FIG. 13 shows HeLa cells incubated with anti-Lamin A/C siRNA in the presence of THCO (A) or Transfast (B) and culture medium (C).

FIG. 14 shows luciferase gene silencing using THCO, PEI, Transfast, and DOTAP as the transfection agent.

FIG. 15 shows the general structure of MFCs.

FIG. 16 shows the synthetic procedure for making EHCO.

FIG. 17 shows the hemolytic activity of MFCs (8.3 μM) and DOTAP (8.3 μM) at pH of 7.4, 6.5 and 5.4, respectively.

FIG. 18 shows the particle size of the siRNA complexes with MFCs. (a) Size of EHCO/siRNA complexes at different N/P ratios. (b) Sizes of siRNA complexes with the MFCs at N/P ratios of 8 and 10.

FIG. 19 shows the autoxidation profile of EHCO in the absence or presence of siRNA based on thiol concentration measured by Ellman's assay.

FIG. 20 shows the EHCO mediated luciferase gene silencing efficiency in U87-luc cells at different N/P ratios.

FIG. 21 shows the MFC-mediated luciferase gene silencing efficiency (bars) and the viability (curves) of cells incubated with MFC/siRNA complexes in U87-Luc cells. Transfection experiments were performed at siRNA concentration of 100 nM (a) or 20 nM (b).

FIG. 22 shows the in vivo Luciferase expression knockdown efficiency of EHCO (MFC)/anti-luc siRNA complexes in U87-luc xenograft mice.

FIG. 23 shows a tumor growth curve of the untreated control mice and mice treated with anti-HIF/EHCO nanoparticles at a siRNA dose of 2 mg/kg in each treatment.

FIG. 24 shows the synthesis of resin-supported synthesis of dithiol-containing monomer possessing primary, secondary and tertiary charge groups.

FIG. 25 shows the synthesis of polydisulfide oligomer 11 (PDS) by oxidative polymerization.

FIG. 26 shows the gel electrophoresis shift assay at the indicated N/P ratios.

FIG. 27 shows size of PDS/DNA and PDS/siRNA complexes at indicated N/P ratios.

FIG. 28a shows transfection efficiency of PDS/DNA complexes in Cos 7 cells at different N/P ratios in the absence or presence of 100 μM CQ in comparison with PEI and Naked DNA. FIG. 28b shows endogenous luciferase gene silencing efficiency by PDS/siRNA or PEI/siRNA complexes in u373-Luc cells under different conditions.

FIG. 29a shows relative cell viability vs. PDS and PEI concentration, and FIG. 29b shows relative cell viability vs. N/P ratio of PDS and PEI of DNA complexes. The cationic PDS exhibited much lower cytotoxicity than PEI.

FIG. 30 shows the synthesis of BN-PEG-Mal.

FIG. 31 shows the synthesis of RGD-PEG-Mal.

FIG. 32 shows a schematic presentation of surface modification of MFC/siRNA or MFC/DNA nanoparticles with maleimide-containing functional molecules.

FIG. 33 shows representative fluorescence images of EHCO/DNA nanoparticles that were functionalized with targeting moiety (BN-PEG-Mal, 2.5% modification degree, A˜C) or without targeting moiety (mPEG-Mal, 2.5% modification degree, D˜F).

FIG. 34 shows the cellular uptake efficiency mediated by targeted nanoparticles (2.5% BN-PEG-Mal modification) via receptor-mediated endocytosis was comparable to that of unmodified nanoparticles via non-specific endocytosis and higher than the pegylated nanoparticles. Pegylation of the nanoparticles reduced non-specific cell uptake.

FIG. 35 shows cellular uptake efficiency mediated by targeted nanoparticles (2.5% RGD-PEG-Mal modification) was significantly higher than that of non-targeted nanoparticles (2.5% mPEG-Mal modification).

FIG. 36 shows that cellular uptake efficiency mediated by 2.5% RGD-PEG-Mal modified targeted nanoparticles decreased in the presence of free RGD.

FIG. 37 shows mice tumor volume based over time upon I.V. administration to mice 2.5% peptide-modified MFC/siRNA nanoparticles (BN-PEG-Mal or RGD-PEG-Mal).

### DETAILED DESCRIPTION

Before the present compounds, compositions, and methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like. “Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or cannot be substituted and that the description includes both unsubstituted lower alkyl and lower alkyl where there is substitution.

The term “alkenyl group” is defined herein as a C₂-C₂₀ alkyl group possessing at least one C═C double bond.

The term “alkyl group” as used herein is a branched or unbranched saturated hydrocarbon group of 1 to 25 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is an alkyl group containing from one to six carbon atoms.

The term “acyl” group as used herein is represented by the formula C(O)R, where R is an organic group such as, for example, an alkyl or aromatic group as defined herein.

The term “alkylene group” as used herein is a group having two or more CH₂ groups linked to one another. The alkylene group can be represented by the formula —(CH₂)ₐ—, where a is an integer of from 2 to 25.

The term “aromatic group” as used herein is any group containing an aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The phrase “nitrogen containing substituent” is defined herein as any amino group. The term “amino group” is defined herein as a primary, secondary, or tertiary amino group. In the alternative, the nitrogen containing substituent can be a quaternary ammonium group. The nitrogen containing substituent can be an aromatic or cycloaliphatic group, where the nitrogen atom is either part of the ring or directly or indirectly attached by one or more atoms (i.e., pendant) to the ring. The nitrogen containing substituent can be an alkylamino group having the formula —R—NH₂, where R is a branched or straight alkyl group, and the amino group can be substituted or unsubstituted.

Variables such as AA¹, AA², A, B, L, R, R¹-R²⁴, a, b, d, m, n, o, p, q, r, s, t, u, v, w, x, y, and z used throughout the application are the same variables as previously defined unless stated to the contrary.

I. Multifunctional Carriers and Methods of Making thereof

Described herein are multifunctional carrier compounds useful for the delivery of nucleic acids to cells. The compounds described herein possess a variety of groups that collectively produce carriers that efficiently deliver nucleic acids to cells. Each of the functional groups will be discussed below.

In one aspect, described herein is a compound comprising the formula I:

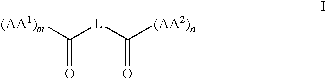

wherein AA¹ and AA comprises one or more amino acids, wherein (AA¹)ₘ and (AA²)ₙ are the same or different sequence;
m and n are an integer from 1 to 50; and
L comprises a group comprising at least one neutral amino group or cationic ammonium group, or the pharmaceutically-acceptable salt, ester, or amide thereof.

The amino acids AA¹ and AA² can be a single amino acid or a plurality of amino acids to form a sequence. In general, the individual amino acids are linked to one another via amide bonds (—NC(O)—). Referring to formula I, the carbonyl group (C═O) bonded to linker L is derived from the carboxylic acid of the terminal amino acid of AA¹ and AA². This is depicted below in the following amino acid sequence: AA^{a}AA^{b}AA^{c}AA^{d}AA^{e}-COOH, where the carboxylic acid group is on amino acid AA^{e}. The amino acids AA¹ and AA² can be composed of the same or different amino acid sequence. When a plurality of amino acids are used in AA¹ and AA², the number can vary depending upon the mechanism of delivering nucleic acids to cells. In one aspect, m and n in formula I are an integer from 1 to 50. Not wishing to be bound by theory, the amino acid sequences can act as pH buffers, nucleic acid complexation agents, amphiphiles, polymerizable monomers, receptor binders, enhance cellular uptake, and facilitate release of the bioactive agent once in the cell.

In one aspect, the compound having the formula I comprises at least one thiol (SH) group. For example, one of the amino acids of (AA¹)ₘ and (AA²)ₙ is a residue of cysteine, homocysteine, or a thiol containing derivative of an amino acid. It is also contemplated that one or more amino acids of (AA¹)ₘ and (AA²)ₙ can be derivatized such that thiol groups are introduced into the sequence. Using techniques known in the art, it is possible to react functional groups present on the amino acid with compounds containing thiol groups. As will be discussed below, once the nucleic acid is complexed with the compound having the formula I to produce nanoparticles, the thiol groups can produce disulfide (S—S) bonds by oxidation to form oligomers and polymers or cross-linking to further stabilize the nanoparticles. The disulfide bonds will be reduced in the cytoplasm to facilitate the release of nucleic acid from the delivery system.

The structure L comprises at least one amino group. The term “amino group” includes a primary amino group, a secondary amino group, a tertiary amino group, and/or aromatic amino group, and/or quaternary ammonium group. The amino group can be neutral or cationic. For example, an amino group can be protonated or treated with an alkylating agent to produce a quaternary (cationic) ammonium group. In the case of substituted amino groups, suitable groups include alkyl and aromatic groups defined herein. In one aspect, a plurality of amino groups is present in structure L. In one aspect, the amino group is part of an alkylene chain, wherein one or more carbon atoms is substituted with nitrogen. In another aspect, the amino group can be pendant to an alkylene chain. Using technology known in the art, it is possible to synthesize various structures with amino groups. The structure of L can vary based upon the properties of the nucleic acids to be delivered to the cells. Examples of suitable groups for L are described below. Not wishing to be bound by theory, the structure L complexes with the nucleic acid to form nanoparticles for the delivery of nucleic acids into cells.

In certain aspects, a hydrophobic group is covalently attached to a least one amino acid of (AA¹)ₘ and (AA²)ₙ. Alternatively, the hydrophobic group can be attached to the structure L. The hydrophobic group can be derived from a saturated or unsaturated C₁-C₂₅ fatty acid (RCOOH, where R is a C₁-C₂₅ alkyl or alkylene group) or C₁-C₂₅ alkyl or alkylene group. Alternatively, the hydrophobic group can be derived from a steroid compound or an aromatic compounds. In one aspect, one or more of the amino groups present on (AA¹)ₘ and (AA²)ₙ have a hydrophobic group bonded to it. Not wishing to be bound by theory, the hydrophobic groups help form compact, stable nanoparticles with the nucleic acids and introduce amphiphilic properties to facilitate pH sensitive escape of nanoparticles from endosomal and lysosomal compartments. This is particularly useful when the compounds are used as in vivo delivery devices.

In other aspects, a targeting group is attached to at least one amino acid of (AA¹)ₘ and (AA²)ₙ, the cationic structure L, or by a thiol group. The targeting agents can be useful in the delivery of nucleic acids into cells. The targeting agent can be a peptide, an antibody, an antibody fragment or one of their derivatives. For example, target-specific peptides can be conjugated directly to the compound or indirectly via a second linker (e.g., polyethylene glycol) prior or during the formation of nanoparticles. Depending upon the selection of the targeting compound, the targeting group can be covalently bonded to either an amino group present on the amino acid or the thiol group or an amino group present on structure L.

In one aspect, the targeting group is indirectly attached to the compound by a linker. Examples of linkers include, but are not limited to, a polyamine group, a polyalkylene group, a polyamino acid group or a polyethylene glycol group. The selection of the linker as well as the molecular weight of the linker can vary depending upon the desired properties. In one aspect, the linker is polyethylene glycol having a molecular weight from 500 to 10,000, 500 to 9,000, 500 to 8,000, 500 to 7,000, or 2,000 to 5,000. In certain aspects, the targeting group is first reacted with the linker in a manner such that the targeting group is covalently attached to the linker. For example, the linker can possess one or more groups that can react with an amino group present on a peptide. The linker also possesses additional groups that react with and form covalent bonds with the compounds described herein. For example, if one or more thiol groups are present on the compound, the linker can possess maleimide groups that readily react with the thiol groups. The selection of functional groups present on the linker can vary depending upon the functional groups present on the compound. In one asepct, the targeting compound is a peptide such as, for example, an RGD peptide or bombesin peptide that is covalently attached to polyethylene glycol.

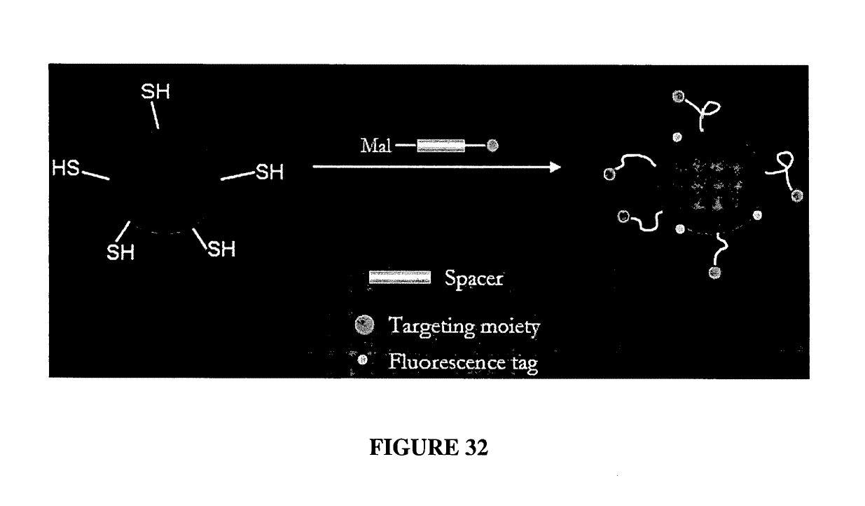

In other aspects, it is also desirable to attach the targeting compound to a nanoparticle produced by the compounds described herein. For example, after a nanoparticle composed of a nucleic acid has been produced using the compounds and techniques described herein, the targeting compound can be attached to the nanoparticle via a linker. One aspect of this approach is depicted in FIG. 32.

In one aspect, the compound represented by formula I comprises the formula II

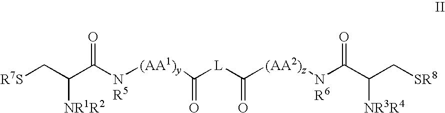

wherein R¹-R⁸ are, independently, hydrogen, an alkyl group, an alkenyl group, an acyl group, an aromatic group, or a hydrophobic group;
AA¹ and AA² are one or more amino acids, wherein (AA¹)_{y} and (AA²)_{z} are the same or different sequence;
y and z are an integer from 0 to 50; and
L comprises a group comprising at least one neutral amino group or cationic ammonium group, or the pharmaceutically-acceptable salt, ester or amide thereof.

Referring to formula II, each end of the compound is capped with cysteine residue. It is contemplated that other thiol groups can be present in formula II, particularly if AA¹ and AA² contain other cysteine residues.

In one aspect, the structure L comprises the formula III

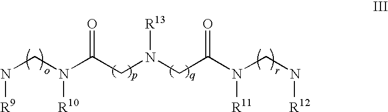

wherein R⁹-R¹² are, independently, hydrogen, an alkyl group, an alkenyl group, an aromatic group;
R¹³ is an alkylamino group or an group containing at least one aromatic amino group; and
o, p, q, and r are, independently, an integer from 1 to 10.

Examples of alkylamino groups are depicted in Formulae IV-VI

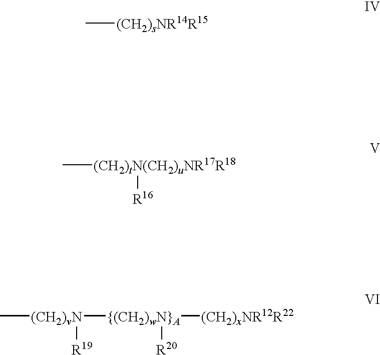

wherein R¹⁴-R²² are, independently, hydrogen, an alkyl group, a nitrogen containing substituent, or a hydrophobic group;
s, t, u, v, w, and x are an integer from 1 to 10; and
A is an integer from 1 to 50.

As shown in formula IV-VI, the number of amino groups can vary. In one aspect, R¹³ in formula III is CH₂NH₂, CH₂CH₂NH₂, CH₂CH₂CH₂NH₂, CH₂CH₂CH₂CH₂NH₂, CH₂CH₂CH₂CH₂CH₂NH₂, CH₂NHCH₂CH₂CH₂NH₂, CH₂CH₂NHCH₂CH₂CH₂NH₂, CH₂CH₂CH₂NHCH₂CH₂CH₂CH₂NHCH₂CH₂CH₂NH₂, CH₂CH₂NHCH₂CH₂CH₂CH₂NH₂, CH₂CH₂NHCH₂CH₂CH₂NHCH₂CH₂CH₂HN₂, or CH₂CH₂NH(CH₂CH₂NH)dCH₂CH₂NH₂, where d is from 0 to 50.

In one aspect, R¹³ comprises an aromatic amino group. The aromatic amino group can include one or more amino groups directly or indirectly attached to the aromatic group as described above. Alternatively, the amino group can be incorporated in the aromatic ring. For example, the aromatic amino group is a pyrrole, an isopyrrole, a pyrazole, imidazole, a triazole, or an indole. In another aspect, the aromatic amino group includes the isoimidazole group present in histidine.

In one aspect, the compound has the formula II and L has the structure depicted in formula III, wherein R¹-R¹² are hydrogen; o, p, q, an r are each 2; y and z are zero; and R¹³ is CH₂CH₂NHCH₂CH₂NHCH₂CH₂NH₂. In another aspect, the compound has the formula II and L has the structure depicted in formula III, wherein R¹ and R³ are a hydrophobic group; R² and R⁴-R¹² are hydrogen; o, p, q, an r are each 2; y and z are zero or one; and R¹³ is CH₂CH₂NHCH₂CH₂NHCH₂CH₂NH₂, CH₂CH₂NH₂, or CH₂CH₂CH₂NHCH₂CH₂CH₂CH₂NHCH₂CH₂CH₂NH₂.

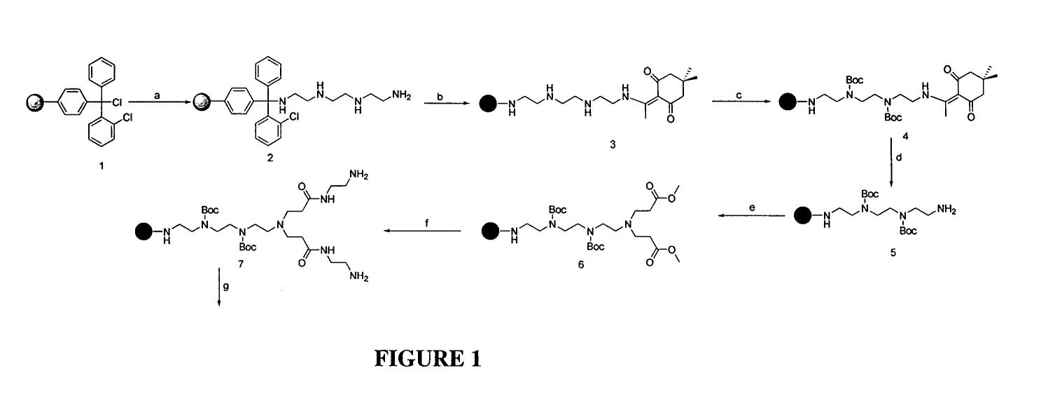

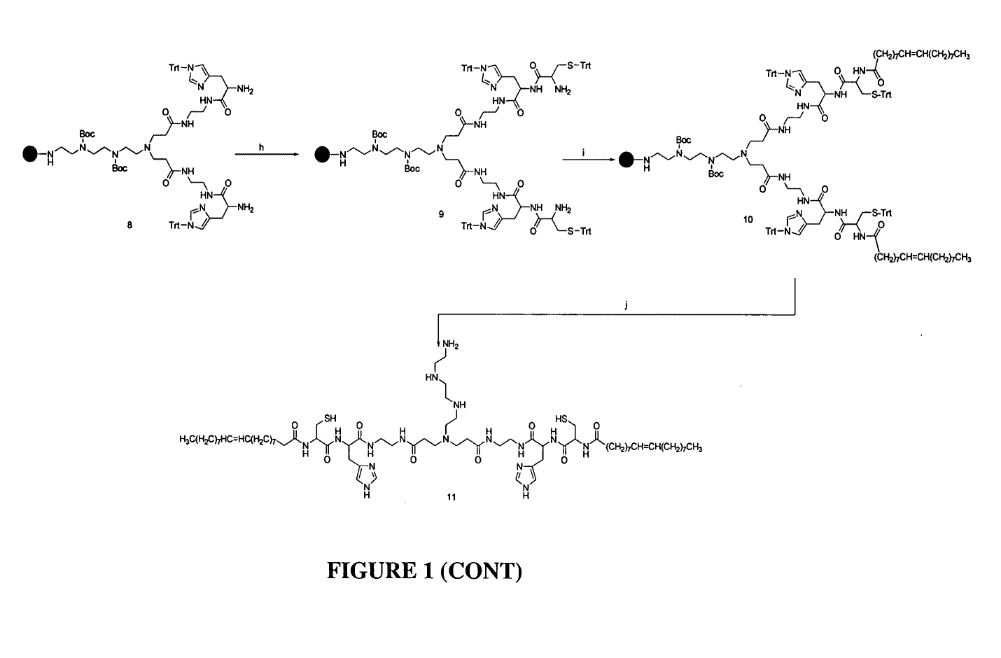

The compounds having the general formula I can be synthesized using solid phase techniques known in the art. FIG. 1 provides an exemplary synthetic procedure for preparing a dithiol compound. In general, the approach in FIG. 1 involves the systematic protection/elongation/deprotection to produce a dithiol compound. The hydrophobic group is produced by reacting oleic acid with the amino group present on the cysteine residue. Although FIG. 1 depicts one approach for producing the compounds of formula I, other synthetic techniques can be used.

The compounds having the formula I have at least two thiol groups that are capable of forming disulfide (S—S) bonds under oxidative conditions. In one aspect, the disulfide compound comprises the formula VII

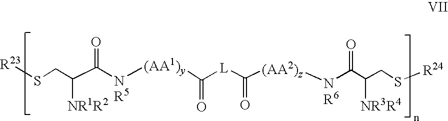

wherein
R¹-R⁶ are, independently, hydrogen, an alkyl group, an alkenyl group, an aromatic group, or a hydrophobic group;
AA¹ and AA² are one or more amino acids, wherein (AA¹)_{y} and (AA²)_{z} are the same or different sequence;
y and z are an integer from 0 to 50;
R²³ and R²⁴ are, independently, hydrogen or a targeting group;
B is an integer from 2 to 10,000;
L comprises a group comprising at least one neutral amino group or cationic ammonium group, or the pharmaceutically-acceptable salt, ester or amide thereof.

The disulfide bonds stabilize the delivery systems and help achieve release of the nucleic acid once it is in the cell. For example, when the nucleic acid is siRNA, the cleavage of disulfide bonds in the siRNA delivery systems in reductive cytoplasm can facilitate cytoplasm-specific release of siRNA. The disulfide compounds will be stable in the plasma at very low free thiol concentration (e.g., 15 μM). When the disulfide compounds are incorporated into target cells, the high concentration of thiols present in the cell (e.g., cytoplasm) will reduce the disulfide bonds to facilitate the dissociation and release of the nucleic acid.

The disulfides having the formula VII can be readily produced by reacting the same or different compounds having the formula I before complexation with nucleic acid or during the complexation in the presence of an oxidant. The oxidant can be air, oxygen or other chemical oxidants. Depending upon the dithiol compound selected and oxidative conditions, the degree of disulfide formation can vary in free polymers or in complexes with nucleic acids. Thus, compounds having the formula I are monomers, and the monomers can be dimerized, oligomerized, or polymerized depending upon the reaction conditions.

Any of the compounds described herein can exist or be converted to the salt thereof. In one aspect, the salt is a pharmaceutically acceptable salt. The salts can be prepared by treating the free acid with an appropriate amount of a chemically or pharmaceutically acceptable base. Representative chemically or pharmaceutically acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. In one aspect, the reaction is conducted in water, alone or in combination with an inert, water-miscible organic solvent, at a temperature of from about 0° C. to about 100° C. such as at room temperature. The molar ratio of the compound to base used is chosen to provide the ratio desired for any particular salts. For preparing, for example, the ammonium salts of the free acid starting material, the starting material can be treated with approximately one equivalent of base to yield a salt.

In another aspect, any of the compounds described herein can exist or be converted to the salt with a Lewis base thereof. The compounds can be treated with an appropriate amount of Lewis base. Representative Lewis bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, THF, ether, thiol reagent, alcohols, thiol ethers, carboxylates, phenolates, alkoxides, water, and the like. In one aspect, the reaction is conducted in water, alone or in combination with an inert, water-miscible organic solvent, at a temperature of from about 0° C. to about 100° C. such as at room temperature. The molar ratio of the compound to base used is chosen to provide the ratio desired for any particular complexes. For example, the ammonium salts of the free acid starting material, the starting material can be treated with approximately one equivalent of chemically or pharmaceutically acceptable Lewis base to yield a complex.

If the compounds possess carboxylic acid groups, these groups can be converted to pharmaceutically acceptable esters or amides using techniques known in the art. Alternatively, if an ester is present on the dendrimer, the ester can be converted to a pharmaceutically acceptable ester using transesterification techniques.

II. Methods of Use

The compounds described herein have numerous applications with respect to the delivery of nucleic acids to a subject. In other aspect, the compounds described herein can be used in gene therapy to deliver genetic materials to cells and tissues.

The nucleic acid can be an oligonucleotide, deoxyribonucleic acid (DNA), ribonucleic acid (RNA), or peptide nucleic acid (PNA). The nucleic acid of interest introduced by the present method can be nucleic acid from any source, such as a nucleic acid obtained from cells in which it occurs in nature, recombinantly produced nucleic acid, or chemically synthesized nucleic acid. For example, the nucleic acid can be cDNA or genomic DNA or DNA synthesized to have the nucleotide sequence corresponding to that of naturally-occurring DNA. The nucleic acid can also be a mutated or altered form of nucleic acid (e.g., DNA that differs from a naturally occurring DNA by an alteration, deletion, substitution or addition of at least one nucleic acid residue) or nucleic acid that does not occur in nature.

In one aspect, the nucleic acid can be a functional nucleic acid. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, siRNA, miRNA, shRNA and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acids can be a small gene fragment that encodes dominant-acting synthetic genetic elements (SGEs), e.g., molecules that interfere with the function of genes from which they are derived (antagonists) or that are dominant constitutively active fragments (agonists) of such genes. SGEs can include, but are not limited to, polypeptides, inhibitory antisense RNA molecules, ribozymes, nucleic acid decoys, and small peptides. The small gene fragments and SGE libraries disclosed in U.S. Patent Publication No. 2003/0228601, which is incorporated by reference, can be used herein.

The functional nucleic acids of the present method can function to inhibit the function of an endogenous gene at the level of nucleic acids, e.g., by an antisense, RNAi or decoy mechanism. Alternatively, certain functional nucleic acids can function to potentiate (including mimicking) the function of an endogenous gene by encoding a polypeptide that retains at least a portion of the bioactivity of the corresponding endogenous gene, and may in particular instances be constitutively active.

Other therapeutically important nucleic acids include antisense polynucleotide sequences useful in eliminating or reducing the production of a gene product, as described by Tso, P. et al Annals New York Acad. Sci. 570:220-241 (1987). Also contemplated is the delivery of ribozymes. These antisense nucleic acids or ribozymes can be expressed (replicated) in the transfected cells. Therapeutic polynucleotides useful herein can also code for immunity-conferring polypeptides, which can act as endogenous immunogens to provoke a humoral or cellular response, or both. The polynucleotides employed according to the present invention can also code for an antibody. In this regard, the term “antibody” encompasses whole immunoglobulin of any class, chimeric antibodies and hybrid antibodies with dual or multiple antigen or epitope specificities, and fragments, such as F(ab)₂, Fab², Fab and the like, including hybrid fragments. Also included within the meaning of “antibody” are conjugates of such fragments, and so-called antigen binding proteins (single chain antibodies) as described, for example, in U.S. Pat. No. 4,704,692, the contents of which are hereby incorporated by reference.

In one aspect, the nucleic acid is siRNA. siRNAs are double stranded RNA molecules (dsRNAs) with approximately 20 to 25 nucleotides, which are generated by the cytoplasmic cleavage of long RNA with the RNase III enzyme Dicer. siRNAs specifically incorporate into the RNA-induced silencing complex (RISC) and then guide the RNAi machinery to destroy the target mRNA containing the complementary sequences. Since RNAi is based on nucleotide base-pairing interactions, it can be tailored to target any gene of interest, rendering siRNA an ideal tool for treating diseases with gene silencing. Gene silencing with siRNAs has a great potential for the treatment of human diseases as a new therapeutic modality. Numerous siRNAs have been designed and reported for various therapeutic purposes and some of the siRNAs have demonstrated specific and effective silencing of genes related to human diseases. Therapeutic applications of siRNAs include, but are not limited to, inhibition of viral gene expression and replication in antiviral therapy, anti-angiogenic therapy of ocular diseases, treatment of autoimmune diseases and neurological disorders, and anticancer therapy. Therapeutic gene silencing has been demonstrated in mammals, which bodes well for the clinical application of siRNA. It is believed that siRNA can target every gene in human genome and has unlimited potential to treat human disease with RNAi.

The nucleic acid can be complexed to the carrier compounds described herein by admixing the nucleic acid and the compound or corresponding disulfide oligomer or polymer. The pH of the reaction can be modified to convert the amino groups present on the compounds described herein to cationic groups. For example, the pH can be adjusted to protonate the amino group. With the presence of cationic groups on the compound, the nucleic acid can electrostatically bond (i.e., complex) to the compound. In one aspect, the pH is from 1 to 7.4. In another aspect, the N/P ratio is from 0.5 to 100, where N is the number of nitrogen atoms present on the compound that can be form a positive charge and P is the number of phosphate groups present on the nucleic acid. Thus, by modifying the compound with the appropriate number of amino groups in the structure L, it is possible to tailor the bonding (e.g., type and strength of bond) between the nucleic acid and the compound. In one aspect, the nucleic acid/carrier complex is a nanoparticle. In one aspect, the nanoparticle has a diameter of about 1000 nanometers or less.

In other aspects, the compounds described herein can be designed so that the resulting nucleic acid nanoparticle escapes endosomal and/or lysosomal compartments at the endosomal-lysosomal pH. For example, the compound forming nanoparticles with nucleic acids can be designed such that its structure and amphiphilicity changes at endosomal-lysosomal pH (5.0-6.0) and disrupts endosomal-lysosomal membranes, which allows entry of the nanoparticle into the cytoplasm. In one aspect, the ability of specific endosomal-lysosomal membrane disruption of the compounds described herein can be tuned by modifying their pH sensitive amphiphlicity by altering the number and structure of protonatable amines and lipophilic groups. For example, decreasing the number of protonatable amino groups can reduce the amphiphilicity of a nanoparticle produced by the compound at neutral pH. In one aspect, the compounds herein have 1 to 50, 1 to 40, 1 to 30, 1 to 20, 1 to 10, 1 to 8, 1 to 6, 1 to 4, or 2 protonatable amino or substituted amino or aromatic amino groups. For example, the amino and/or substituted and/or imidazolyl amino groups can be present on the linker L in formulae I, II, or VII. In one aspect, the compounds described herein include at least one histidine residue. In other aspects, the compounds include 2, 3, 4, 5, 6, 7, 8, 9, or 10 histidine residues. Thus, the pH-sensitive amphiphilicity of the compounds and nanoparticles produced by the compounds can be used to fine-tune the overall pKa of the nanoparticle. Low amphiphilicity of the nanoparticles at physiological pH can minimize non-specific cell membrane disruption and nonspecific tissue uptake of the nucleic acid/MFC system. In certain aspects, it is desirable that the carriers have low amphiphilicity at the physiological pH and high amphiphilicity at the endosomal-lysosomal pH, which will only cause selective endosomal-lysosomal membrane disruption with the nanoparticles.

The surface of the nanoparticle complexes can be modified by, for example, covalently incorporating polyethylene glycol by reacting unpolymerized free thiol of the nanoparticle to reduce non-specific tissue uptake in vivo. For example, PEG-maleimide reacts rapidly with free thiol groups. The molecular weight of the PEG can vary depending upon the desired amount of hydrophilicity to be imparted on the carrier. PEG-modification of the carrier can also protect nanoparticles composed of the nucleic acid from enzymatic degradation upon uptake by the cell (e.g., endonucleases). Targeting agents, including peptides, proteins, antibodies or antibody fragment, can also be incorporated into the nanoparticle complexes during the preparation of the complexes to enhance the delivery specificity and efficiency of the genetic materials to the target cells. Polyethylene glycol can be used as the spacer to conjugate targeting agents to the nanoparticle complexes.

The compounds described herein can be used to introduce a nucleic acid into a cell. The method generally involves contacting the cell with a complex, wherein the nucleic acid is taken up into the cell. In one aspect, the compounds described herein can facilitate the delivery of DNA or RNA as therapy for genetic disease by supplying deficient or absent gene products to treat any genetic disease or by silencing gene expression. Techniques known in the art can used to measure the efficiency of the compounds described herein to deliver nucleic acids to a cell.

The term “cell” as used herein is intended to refer to well-characterized homogenous, biologically pure populations of cells. These cells may be eukaryotic cells that are neoplastic or which have been “immortalized” in vitro by methods known in the art, as well as primary cells, or prokaryotic cells. The cell line or host cell is preferably of mammalian origin, but cell lines or host cells of non-mammalian origin may be employed, including plant, insect, yeast, fungal or bacterial sources.

In one aspect, the cell comprises stem cells, committed stem cells, differentiated cells, primary cells, and tumor cells. Examples of stem cells include, but are not limited to, embryonic stem cells, bone marrow stem cells and umbilical cord stem cells. Other examples of cells used in various embodiments include, but are not limited to, osteoblasts, myoblasts, neuroblasts, fibroblasts, glioblasts, germ cells, hepatocytes, chondrocytes, keratinocytes, smooth muscle cells, cardiac muscle cells, connective tissue cells, glial cells, epithelial cells, endothelial cells, hormone-secreting cells, cells of the immune system, and neurons.

Atypical or abnormal cells such as tumor cells can also be used herein. Tumor cells cultured on substrates described herein can provide more accurate representations of the native tumor environment in the body for the assessment of drug treatments. Growth of tumor cells on the substrates described herein can facilitate characterization of biochemical pathways and activities of the tumor, including gene expression, receptor expression, and polypeptide production, in an in vivo-like environment allowing for the development of drugs that specifically target the tumor.

The complexes (i.e., nanoparticles) described above can be administered to a subject using techniques known in the art. For example, pharmaceutical compositions can be prepared with the complexes. It will be appreciated that the actual preferred amounts of the complex in a specified case will vary according to the specific compound being utilized, the particular compositions formulated, the mode of application, and the particular situs and subject being treated. Dosages for a given host can be determined using conventional considerations, e.g., by customary comparison of the differential activities of the subject compounds and of a known agent, e.g., by means of an appropriate conventional pharmacological protocol. Physicians and formulators, skilled in the art of determining doses of pharmaceutical compounds, will have no problems determining dose according to standard recommendations (Physicians Desk Reference, Barnhart Publishing (1999).

Pharmaceutical compositions described herein can be formulated in any excipient the biological system or entity can tolerate. Examples of such excipients include, but are not limited to, water, saline, Ringer's solution, dextrose solution, Hank's solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin and benzyl alcohol.

Pharmaceutical carriers are known to those skilled in the art. These most typically would be standard carriers for administration to humans, including solutions such as sterile water, saline, and buffered solutions at physiological pH.

Molecules intended for pharmaceutical delivery can be formulated in a pharmaceutical composition. Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically, including ophthalmically, vaginally, rectally, intranasally. Administration can also be intravenously or intraperitoneally. In the case of contacting cells with the nanoparticlar complexes of nucleic acid and MFC described herein, it is possible to contact the cells in vivo or ex vivo.

Preparations for administration include sterile aqueous or non-aqueous solutions, suspensions, and emulsions. Examples of non-aqueous carriers include water, alcoholic/aqueous solutions, emulsions or suspensions, including saline and buffered media. Parenteral vehicles, if needed for collateral use of the disclosed compositions and methods, include sodium chloride solution, Ringer's dextrose, dextrose and sodium chloride, lactated Ringer's, or fixed oils. Intravenous vehicles, if needed for collateral use of the disclosed compositions and methods, include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Dosing is dependent on severity and responsiveness of the condition to be treated, but will normally be one or more doses per day, with course of treatment lasting from several days to several months or until one of ordinary skill in the art determines the delivery should cease. Persons of ordinary skill can easily determine optimum dosages, dosing methodologies and repetition rates. It is understood that any given particular aspect of the disclosed compositions and methods can be easily compared to the specific examples and embodiments disclosed herein, including the non-polysaccharide based reagents discussed in the Examples. By performing such a comparison, the relative efficacy of each particular embodiment can be easily determined. Particularly preferred compositions and methods are disclosed in the Examples herein, and it is understood that these compositions and methods, while not necessarily limiting, can be performed with any of the compositions and methods disclosed herein.

### Examples

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

I. Multifunctional Dithiol Compounds and Characterization thereof

A. General

2-Chlorotrityl chloride resin (1.1 mmol/g), N-fluorenylmethoxycarbonyl-N-im-trityl-L-histidine (Fmoc-His(Trt)-OH), N-fluorenylmethoxycarbonyl-S-trityl-L-cysteine (Fmoc-Cys(Trt)-OH), 2-Acetyldimedone (Dde-OH), N-Hydroxybenzotriazole (HOBt) and 2-(1H)-Benzotriazole-1-yl)-1,1,3,3-tetramethyluronium tetrafluoroborate (TBTU) were purchased from EMD Biosciences (San Diego, Calif.). Ethylene diamine, pentaethylenehexamine, spermine, triethylenetetramine, N,N-diisopropylethyleneamine (DIPEA), methyl acrylate, 1,2-ethylenediamine, hydrazine, oleic acid, triisobutylsilane (TIBS), 1,2-ethanedithiol (EDT), 4-dithiothreitol (DTT) piperidine, trifluoroacetic acid (TFA) were purchased from Lancaster (Windham, N.H.). Dimethyl sulfoxide (DMSO), N,N-dimethylformamide (DMF) and dimethanechloride (DCM) were extra-dry solvents and they were purchased from Acros (Pittsburgh, Pa.). For solid-phase synthesis, except other mentioned, the reactions were performed in ISOLUTE column reservoirs equipped with caps and frits (Charlottesville, Va.). The amines were purified by distillation under reduced pressure before they were used for synthesis. All other materials and solvents were used without additional purification. 5,5′-Dithiobis-(2-nitrobenzoic acid) (DTNB) was purchased from Pierce Inc. (Rockford, Ill.).

Branched PEI (Mw=25 KDa), N-(2,3-dioleoyloxy-1-propyl)trimethylammonium methyl sulfate (DOTAP), bovine serum albumin(BSA), 2,5-Diphenyl-3-(4,5-dimethyl-2-thiazolyl)tetrazolium Bromide (MTT), FITC tagged Goat anti-Mouse IgG antibody were purchased from Sigma-Aldrich (St. Louis, Mo.). MPEG-Mal-5000 was purchased from Nektar (Huntsville, Ala.). 5,5′-dithiobis-(2-nitrobenzoic acid) (DTNB) was purchased from Pierce Inc. (Rockford, Ill.). TransFast™ was purchased from Promega that is composed of N,N-[bis-(2-hydroxyethyl)-N-methyl-N-[2,3-di(tetradecanoyloxy)propyl] ammonium iodide and dioleoyl phosphatidylethanolamine (DOPE). Mouse Anti-Lamin A/C monoclonal antibody was purchased from Abcam Inc. (Cambridge, Mass.). gWiz™ reporter plasmid encoding luciferase and green fluorescence protein were purchased from Aldevron (Fargo, N. Dak.). siRNA targeting lamin A/C was purchased from Ambion, Inc. (Austin, Tex.). The sequence of antisense of a siRNA is 5′-UGUUCUUCUGGAAGUCCAGdTdT-3′ and that of sense is 3′-dTdTACAAGAAGACCU UCAGGUC-5′. siRNA targeting Firefly Luciferase was purchased from Dharmacon (Chicago, Ill.). The sequence of antisense is 5′-UCGAAGUACUCAGCGUAAGdTdT-3′ and that of sense is 3′-dTdTAGCUUCAUGAGUCGCAUUC-5′. Silencer® Negative Control siRNA (Ambion) was used as non-specific siRNA control.

High Performance Liquid Chromatography (HPLC) was carried out using an Agilent 1100 Series Purificaton System. Final product was purified with preparative HPLC, which is equipped with ZORBAX PrepHT C-18 column using a gradient mobile phase (A: 0.05% TFA in water, B: 0.05% TFA in acetonitrile) and a flow rate with 5 mL/min was used. ¹H NMR spectra were obtained using a Varian Mercury 400 (Palo Alto, Calif.). The molecular weight of compound was determined by matrix-assisted laser desorption ionization (MALDI) time-of-flight (TOF) mass spectrometry.

B. Synthesis and Purification of Monomeric Multifunctional Carrier Compound THCO (FIG. 1)

1-[2-chlorotrityl-amino]-1,4,7,10-Tetraazadecane resin (Resin 2). 2-Chlorotrityl chloride resin (300 mg, 1.1 mmol/g) was extensively washed with dry DCM. A solution of triethylenetetramine (1 mL) and DIPEA (64 mg) in DCM was added to the resin, and the suspension was shaken for 2 h. The solvent was drained and then washed with DCM and MeOH. The resin was further shaken with 10 mL DCM/MeOH/DIPEA (17/2/1, v/v/v) for 20 min. The resulted resin 2 was extensively washed with DMF, DCM and dried under reduced pressure.

Selective protection and deprotection of polyamine with 2-acetyldimedone (Dde-OH) and tert-butyloxycarbonyl (Boc) groups. A solution of 2-acetyldimedone (Dde-OH, 2 g) in 10 mL DMF was added to the resin 2. The suspension was shaken at room temperature for 12 h. The resin was extensively washed with DMF and DCM, and solvent was drained to give resin 3. The resin 3 was suspended in the solution of Boc₂O (10 g) in 15 mL DCM. The mixture was shaken at room temperature for 4 h. The resulted resin was extensively washed with DMF and DCM THEN dried under reduced pressure to afford resin 4. Next, the resin 4 was suspended in the solution of 2% hydrazine in DMF for 15 min. This step was repeated three times to ensure complete deprotection of Dde groups. The resulted resin was extensively washed with DMF and DCM to give resin 5. It was dried under reduced pressure for the next step.

Elongation sequentially with methyl acrylate, 1,2-ethylenediamine, N-α-Fmoc-N-im-trityl-L-histidine and N-α-Fmoc-S-trityl-L-cycteine. Dry resin 5 was transferred into 100 mL round flask, and methyl acrylate (50 mL) and DMF (10 mL) were added. The reaction was carried out in rotary evaporator at 50° C. with continuous shaking. The reaction was monitored by Kaiser ninhydrin test till complete consumption of primary amine. The resulted resin 6 was extensively washed with DMF, MeOH, DCM and dried under reduced pressure. For the next step, a solution of 1,2-ethylenediamine (50 mL) in DMF (10 mL) was added to the resin 6. The reaction has been performed in rotary evaporator at 50° C. with continuous shaking for 5 days. The resin was extensively washed with DMF, MeOH, DCM and dried under reduced pressure to give resin 7. The resin was transferred back in ISOLUTE column reservoirs equipped with caps and frits for next solid-phase reactions. A solution of activated N-α-Fmoc-N-im-trityl-L-histidine (2 g) with TBTU/HOBt/DIPEA in DMF was added to resin 7 and the coupling reaction was continued for 2 h. The quality of coupling was followed by Kaiser test. The resin was subjected to washing cycle and Fmoc protecting groups were removed by suspension resin in 20% piperidine in DMF (20 min×3) to afford resin 8. The resin was then extensively washed with DMF and DCM, dried under reduced pressure for the next step. Subsequently, a solution of activated N-α-Fmoc-S-trityl-L-cycteine (2 g) with TBTU/HOBt/DIPEA in DMF was added to resin 8 and the coupling reaction was continued for 2 h. The quality of coupling was followed by Kaiser test. The resin was extensively washed and Fmoc protecting groups were removed with 20% piperidine in DMF. The resulted resin 9 was then extensively washed with DMF, MeOH and DCM for the next step.

Elongation with Oleic Acid. A solution of activated oleic acid (2 g) with TBTU/HOBt/DIPEA in DMF was added to resin 9 and the coupling reaction was continued for 2 h. The quality of coupling was followed by Kaiser test. The resin was extensively washed and Fmoc protecting groups were removed with 20% piperidine in DMF. The resulted resin 10 was then extensively washed with DMF, MeOH and DCM for the next step.

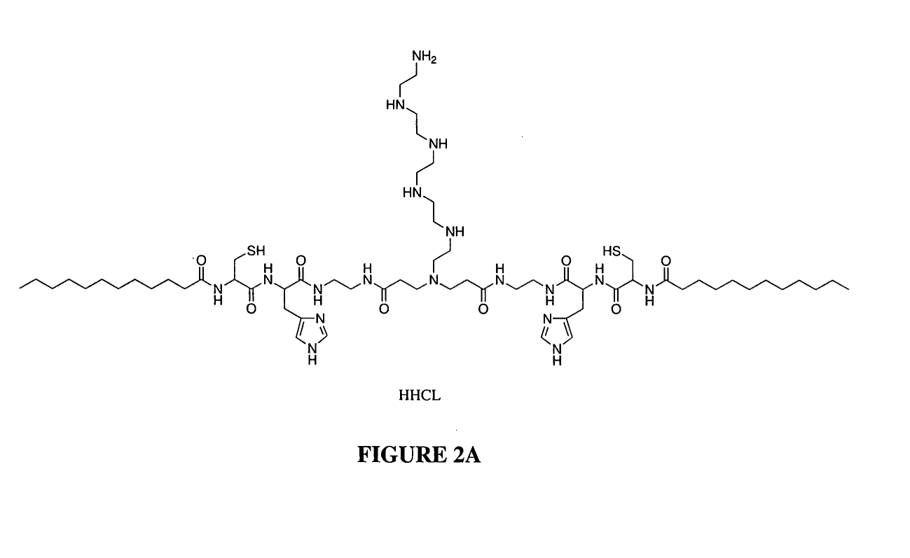

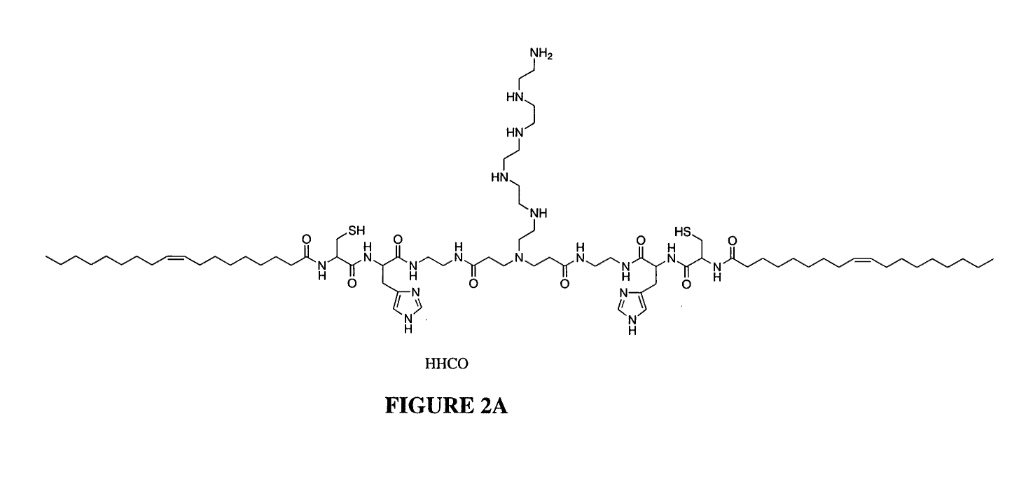

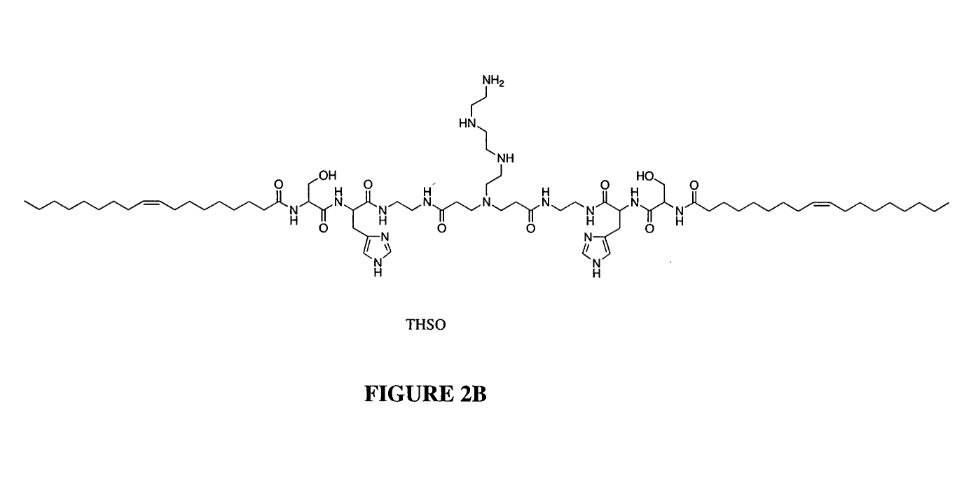

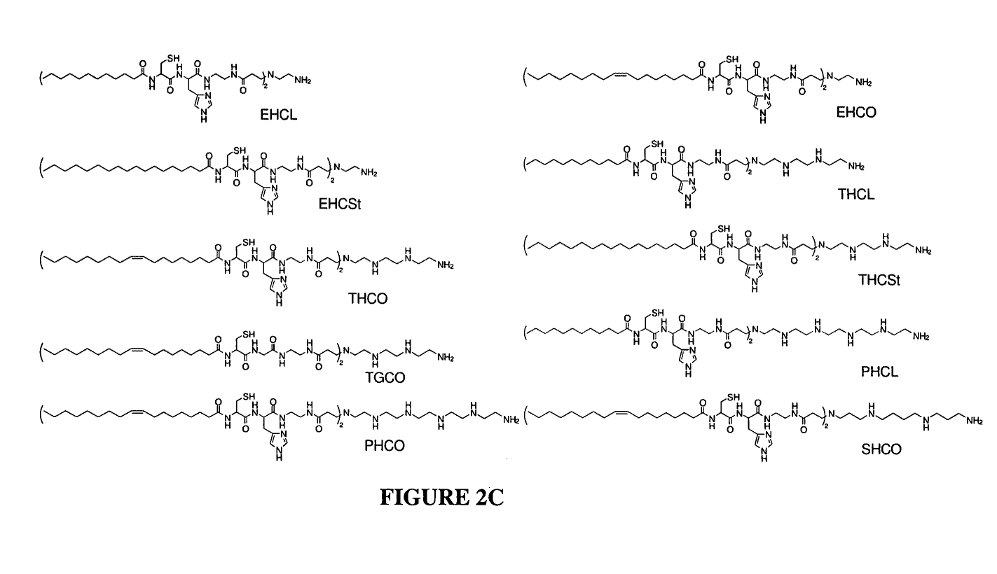

Cleavage and Deprotection of Monomeric Multifunctional Carriers from the Resin. The resin 10 was suspended in a solution of TFA/H₂0/EDT/TIBS (94/2.5/2.5/1). After shaking for 3 h at room temperature, the solution was collected and concentrated under reduced pressure. The monomeric multifunctional compound 11 (THCO; FIG. 1) was washed with cold diethyl ether (40 mL×5) and dried. The compound was further purified by preparative HPLC and appropriate fractions were collected and dried by lyopholization. MS m/z calcd for C₃₄H₆₂N₁₆O₆S₂(M+H)⁺ 1383.94, found 1383.75. A similar procedure was used to produce other MFCs shown in FIG. 2.

C. Physiochemical and Biological Properties of THCO and THCO/Nucleic Acid Complexes

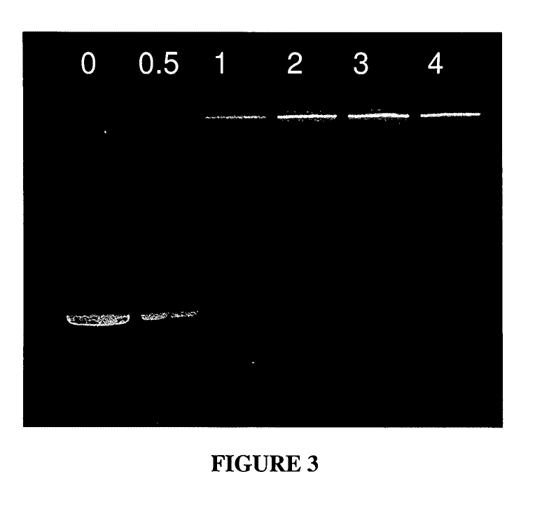

Gel Electrophoresis Shift Assay. The ability of the multifunctional compound THCO to bind DNA was examined by gel electrophoresis. Agarose gel (0.8%, w/v) containing 0.5 μg/mL ethidium bromide was prepared in TAE (Tris-Acetate-EDTA) buffer. DNA (10 μL, 0.1 μg/μL) was mixed with an equal volume of lipid solution at predetermined N/P ratios (N=nitrogens that can be protonated, P=phosphate groups on DNA) and incubated for 30 min before use. 10 μL of each sample was mixed with 2 μL of 6× loading dye and the mixture was loaded onto an agarose gel. The gel was run at 100V for 60 min. The location of DNA bands was visualized on a UV illuminator. FIG. 3 shows the THCO retarded DNA migration in gel at N/P ratio of 1 and above. THCO exhibited strong DNA bind affinity, which can retard DNA migration in the agarose gel at N/P ratio of 1 and above.

Hemolysis assay. THCO (16.7 μM), DOTAP (16.7 μM) and Triton X-100 (1%, w/v) were dissolved in phosphate buffered saline (PBS) at a starting pH of 7.4, 6.5, or 5.4 as stock solutions. A rat was sacrificed and blood was obtained by cardiac puncture. Erythrocytes (RBC) were isolated by centrifugation at 1500 g for 10 min at 4° C. The cell pellet was resuspended into a 2% (w/v) RBC solution with prechilled PBS and then seeded (100 μL) into a 96-well plate. 100 μL of testing samples were added to RBC solutions and the plate was incubated for 1 h at 37° C. The absorbance of the supernatant from each sample was measured at 550 nm using a micro-plate reader.

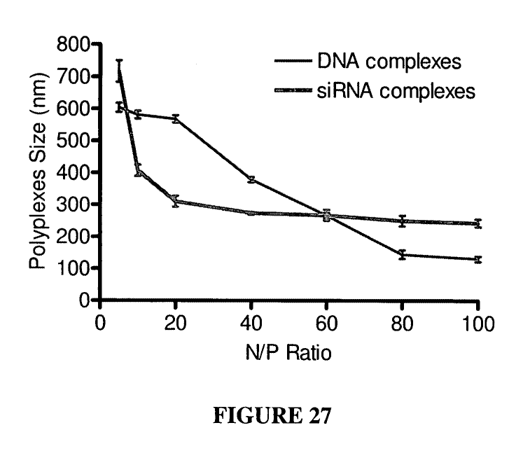

Particle Size Analysis. Samples were prepared by mixing 5.0 μg of plasmid DNA or siRNA with appropriate amount of materials in dust-free water and analyzed using a Brookhaven Instruments BI-200SM system equipped with a 5 mW helium neon laser with a wavelength output of 633 nm. The effective diameter and population distribution were computed from the diffusion coefficient using functions supplied by the instrument. Measurements were made at 25° C. at an angle of 90° and each sample was analyzed in triplicate. The formation of nanoparticles of THCO with siRNA was investigated by dynamic light scattering (DLS) using DOTAP, a non-polymerizable surfactant, as a control. The particle size of THCO/siRNA complexes at an N/P ratio of 8 was approximately 130 nm in diameter in dust free water, while DOTAP/siRNA complexes had a size of about 210 nm in diameter. Both complexes were under the reported cut-off size of 250 nm for efficient cellular uptake. The particle size of siRNA/THCO was much smaller than the siRNA complexes with a polydisulfide of 1,4,7-triazanonylimino-bis[N-(cysteinyl-histinyl)-1-aminoethyl)propionamide at such a low N/P ratio (FIG. 27). Not wishing to be bound by theory, the hydrophobic residues in THCO facilitate the formation of small and compact nanoparticles with siRNA. The nanoparticles of THCO/siRNA complexes were also more stable in 10% FBS medium than those of DOTAP/siRNA complexes. The size of the THCO/siRNA complexes changed from 134 nm at 30 minutes of incubation with the medium to 238 nm at 2 hours of incubation, while the size of DOTAP/siRNA complexes increased from 211 nm at 30 minutes to 727 nm at 2 hours under the same condition, possibly due to particle aggregation.

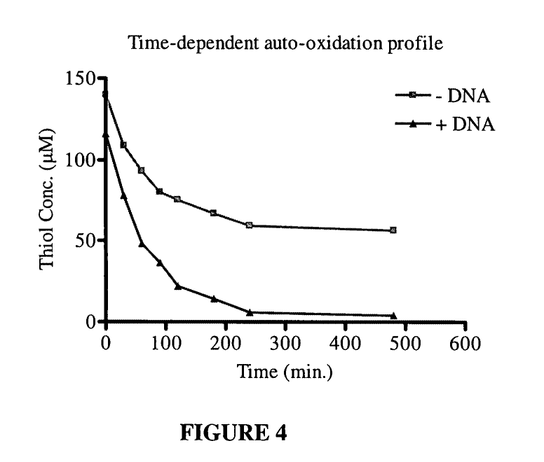

Time dependent oxidation profile. THCO was diluted from a stock solution (2 mg/mL) to an initial theoretical thiol concentration of 180 μM in 4 mL of Tris buffer (10 mM pH 8.0). For each predetermined time point, a 0.2 mL aliquot was removed and mixed with 0.2 mL Ellman's stock solution (2 mM DTNB in 50 mM NaOAc solution). The remaining free thiol concentration was measured by UV-Vis spectrophotometer (Cary-300 Bio). For the complexes experiment, gWiz plasmid DNA was added to a final concentration of 90 μM phosphate before addition of the detergent (N/P=6). FIG. 4 shows the time dependent oxidation profile of THCO by Ellman's reagent.

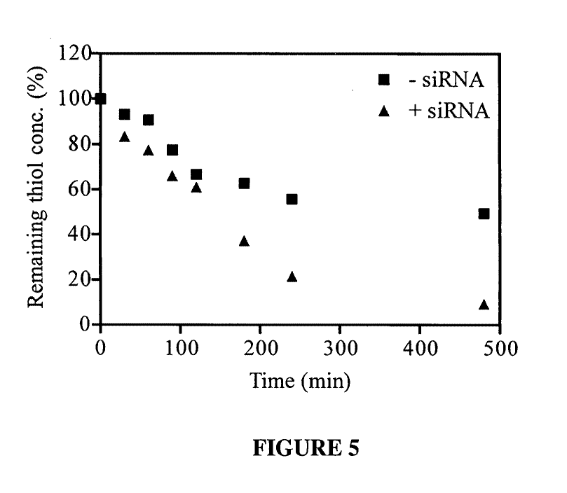

In another experiment, THCO was diluted from a stock solution to an initial thiol concentration of 100 μM in Tris buffer (10 mM pH 8.0). THCO working solution was mixed with or without 10 μg siRNA (N/P=10, where N=nitrogens in the THCO that can be protonated, P=phosphate groups in siRNA). For each predetermined time point, an aliquot was removed and mixed with equal volume of Ellman's solution (2 mM DTNB in 50 mM NaOAc solution), and the thiol concentration was determined by UV-Vis spectrophotometry. The polymerization of the carriers was confirmed by decrease of free thiol concentration in the THCO/siRNA complexes over the time as determined using Ellman's reagent. As shown in FIG. 5, the free thiol concentration decreased more rapidly in the presence of siRNA than that in the absence of siRNA. The complexation with siRNA may facilitate the oxidative polymerization by oxygen.

Cell culture. HeLa and U87 cells were obtained from ATCC (American Type Culture Collection, Rockville, Md., USA) and maintained at 37° C. in a humidified 5% CO₂ atmosphere. Growth medium was supplemented with fetal bovine serum (10%), streptomycin (100 μg/mL) and penicillin (100 units/mL). U-87 cells with constitutive firefly luciferase expression (U87-Luc) were obtained from Huntsman Cancer Institute, University of Utah. U87-Luc was generated by infecting cells with recombinant retroviruses containing a luciferase gene. Cells were maintained in minimal essential medium (ATCC) containing 10% FBS, G418 (300 μg/mL), streptomycin (100 μg/mL) and penicillin (100 units/mL).

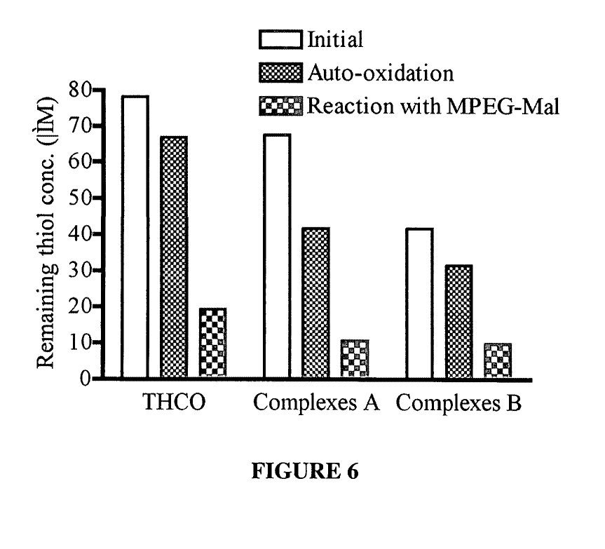

Competition of thiol consumption (auto-oxidation vs. maleimide). THCO or its DNA complex nanoparticles (N/P=6, incubation for 30 min, complexes A; incubation for 60 min, complexes B) were prepared as described above. At predetermined time points, a 0.2 mL solution was mixed with 16 μL of MPEG-Mal-5000 stock solution (10 μg/μL in DMSO) and the mixture was incubated at room temperature for 30 minutes followed by quantification of remaining free thiol concentration. FIG. 6 shows the competition of thiol consumption of THCO between auto-oxidation and reactivity with maleimide.

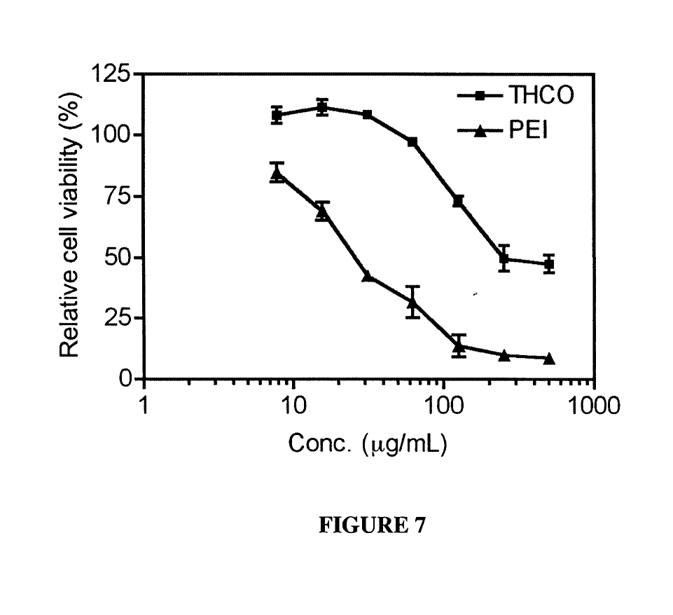

Cytotoxicity assay. Cytotoxicity of THCO in comparison with PEI was evaluated using MTT assay. MB-231 cells were seeded in a 96-well plate 24 h before the assay at a density of 10,000 cells/well. The cells were incubated with 200 μL of complete L-15 containing THCO at different concentrations. After 4 h, the medium in each well was replaced with 100 μL of fresh complete medium. Then 25 μL MTT solution in PBS was added and cells were incubated for another 2 hours. After removing media, 200 μL of DMSO was added to the wells and cells were incubated at 37° C. for 5 min. The optical densities were measured at 570 nm using a micro-plate reader (Model 550, Bio-Rad Lab. Hercules, Calif.). Calculate the relative cell viability by ([Abs]ₛₐₘₚₗₑ-[Abs]_{blank})/([Abs]_{control}-[Abs]_{blank})×100%. FIG. 7 shows the relative cell viability (%) vs. concentration of THCO and PEI. FIG. 7 shows that THCO exhibited lower cytotoxicity in MB-231 cells when compared to PEI.

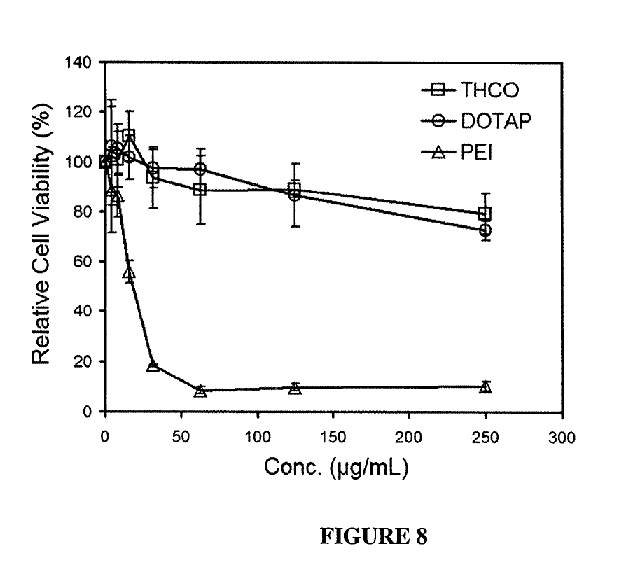

In another study, U87 cells were seeded in a 96-well plate 24 h before the assay at a density of 10,000 cells/well. The cells were incubated with 200 μL of medium containing THCO at different concentrations and incubate for 24 h. Then 25 μL MTT solution in PBS was added and cells were incubated for another 2 hours. After removing media, 200 μL of DMSO was added to the wells and cells were incubated at 37° C. for 5 min. The optical densities were measured at 570 nm using a micro-plate reader (Model 550, Bio-Rad Lab. Hercules, Calif.). The relative cell viability was calculated as described above. THCO exhibited much lower cytotoxicity than PEI that is regarded as a leading transfection agent. The viability of U87 cells incubated with THCO was 79±8% at a concentration of 250 μg/mL as determined the MTT assay, slightly higher than that of DOTAP (72±4%) (FIG. 8). In contrast, only 10±1% of cells incubated with PEI remained viable at the concentration of 62.5 μg/mL or higher, suggesting THCO as a safer carrier than PEI.

Fluorescence labelling and PEG modification of THCO/siRNA complexes. All of conjugation experiments were performed in 10 mM Tris buffer (pH 7.0). 20 μg siRNA was mixed with 80 nmol of THCO followed by adding 0.8 nmol fluorescein-5-maleimide. For fluorescein labelled pegylated nanoparticles, 0.5% or 2.5% mPEG₅₀₀₀-Mal (mole ratio based on MFC) was added 5 minutes after mixing THCO, siRNA and fluorescein-5-maleimide. After 2 h incubation in dark, free maleimide derivates were removed by ultrafiltration (Nanosep®, MWCO=100K, 5000 g, 5 min) and the nanoparticles were recovered for further experiments.

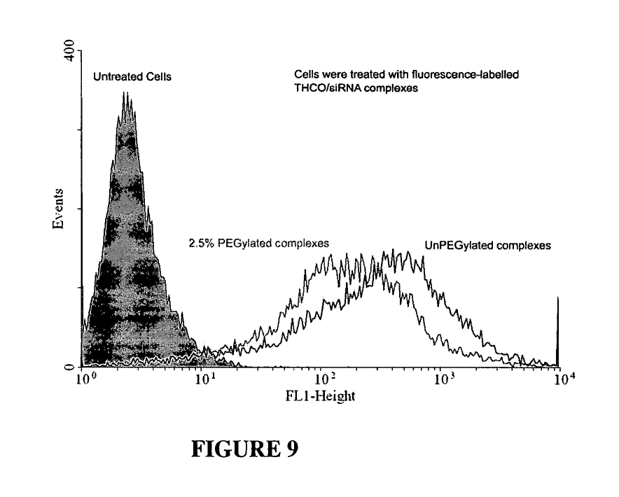

Cellular uptake. Approximately 500,000 U87 cells were plated per well in a 6-well plate 24 hours prior to study. Fluorescence-tagged nanoparticles described above were incubated with cells (5 μg siRNA per well) at 37° C. After 2 h the medium was removed by aspiration and the cells were washed twice with cold phosphate buffered saline (PBS) and then trypsinized. The cells were collected and fixed with 2% polyformaldehyde in PBS at 4° C. for 20 min. Samples were analyzed on a FACSCalibur flow cytometer (BD Biosciences). Results were analyzed using WinMDI software version 2.9. Flow cytometry showed that the unpegylated THCO/siRNA complexes had higher cellular uptake in U87 cells than the PEGylated nanoparticles. Pegylation of nanoparticles most likely reduced non-specific cellular interaction, resulting in lower cellular uptake (FIG. 9).

D. In Vitro Delivery of Nucleic Acids by THCO

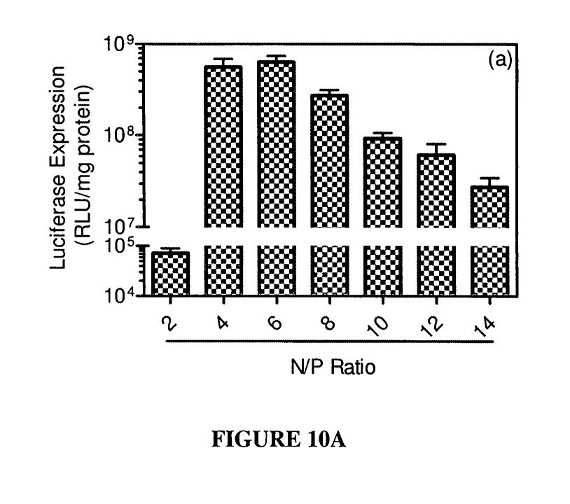

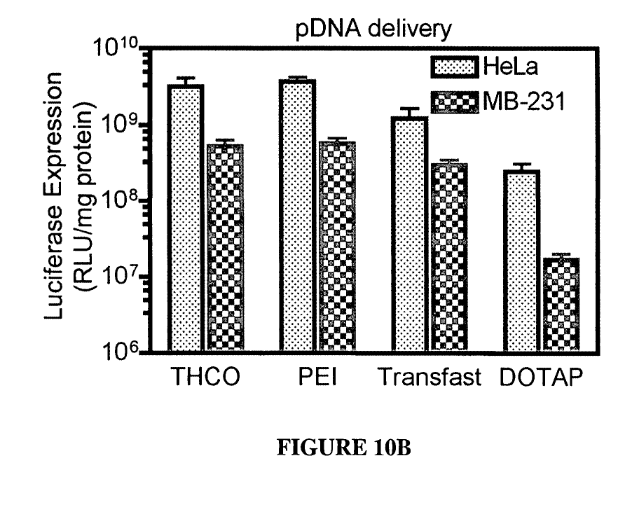

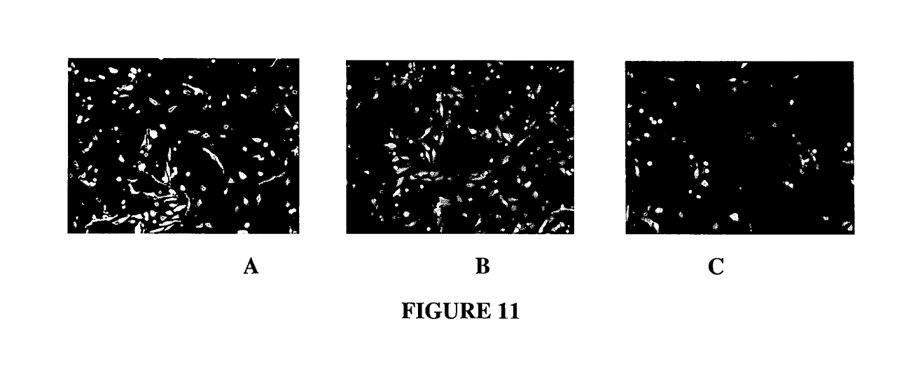

MFC-mediated delivery of plasmid DNA encoding firefly luciferase or GFP. MDA-MB-231 cells were seeded 24 hours prior to transfection into a 24-well plate. At the time of transfection, the medium in each well was replaced with serum free medium. The complexes of THCO/DNA at different N/P ratios were incubated with the cells for 4 hours at 37° C. The medium was then replaced with 1 mL of fresh complete medium and cells were incubated for an additional 44 hours. All the transfection tests were performed in triplicate wells. In the case of plasmid DNA encoding firefly luciferase, after the incubation, cells were treated with 200 μL of cell lysis buffer (Promega Co., Madison, Wis.). The luciferase activity in cell extracts was measured using a luciferase assay kit (Promega Co., Madison, Wis.) on a luminometer for 10 seconds (Lumat 9605, EG&G Wallac). The relative light units (RLU) were normalized against protein concentration in the cell extracts, measured by a BCA protein assay kit (Pierce, Rockford, Ill.). Luciferase activity was expressed as relative light units (RLU/mg of protein in the cell lysate). THCO has high transfection efficiency as shown FIG. 10B. FIG. 10A indicates that a N/P ratio of 4 and 6 provides optimum expression. In the case of plasmid DNA encoding green fluorescence protein, the GFP expression was visualized by fluorescence microscopy (FIG. 11).

THCO-mediated delivery of siRNA targeting a luciferase reporter gene. U87-Luc cells were seeded 24 hours prior to transfection into a 96-well plate at a density of 5000 cells/well. At the time of siRNA transfection, the medium in each well was replaced with fresh serum-free medium. 20 nM anti-luciferase siRNA or non-specific siRNA was complexed with THCO or Transfast™ or DOTAP and incubated for 30 min before use. The transfection by Transfast or DOTAP was performed according to the manufacture's instruction. Complexes were incubated with the cells for 4 hours at 37° C. The medium was then replaced with 100 μL of fresh complete medium and cells were incubated for additional 44 hours. All the transfection tests were performed in triplicate wells. After the incubation, cells were washed with pre-warmed PBS and treated with 200 μL cell lysis buffer and then subjected to a freezing-thawing cycle. Cellular debris was removed by centrifugation at 14,000 g for 5 minutes. The luciferase activity in cell lysate (20 μL) was measured using a luciferase assay kit (100 μL luciferase assay buffer) on a luminometer for 10 seconds (Lumat 9605, EG&G Wallac). The gene silencing efficiency was normalized against the luciferase expression of untreated cells. The siRNA delivery efficiency was also similarly evaluated in 10% FBS medium.

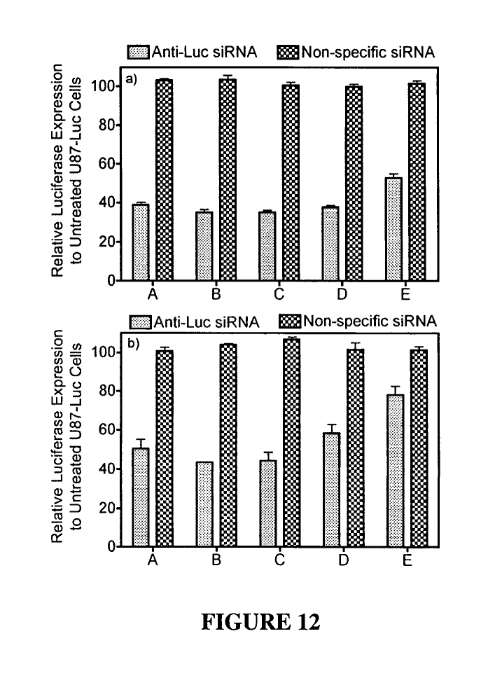

THCO mediated 60-70% gene silencing efficiency with broad N/P ratios from 4 to 16, comparable to Transfast™ (62%), better than that of DOTAP (47%). FIG. 12 shows the gene silencing efficiency of THCO/siRNA complexes and pegylated THCO/siRNA complexes at an N/P ratio of 8, and the control complexes with Transfast and DOTAP in the serum free medium or 10% FBS medium. High gene silencing efficiency was observed for the THCO/siRNA complexes and pegylated complexes without helper lipid DOPE, which is an advantageous feature for the THCO as a ready-to-use carrier as compared to commercially available liposomes or lipid-based transfection agents. Pre-formulation of incorporating DOPE or cholesterol is often required for the typical lipid based carriers to achieve high transfection efficiency, but it is not necessary for THCO.

THCO/siRNA complexes modified with 0.5% and 2.5% mPEG₅₀₀₀-Mal resulted in slightly higher gene silencing efficiency in the serum-free medium and significantly higher efficiency in 10% FBS medium than un-modified THCO/siRNA complexes and Transfast complexes, even if the pegylated MFC/siRNA complexes showed less cellular uptake. Pegylated MFC/siRNA complexes resulted in approximately 56% gene silencing efficiencies in the 10% FBS medium, a slight decrease from that (65%) in serum-free medium. Significant decrease of gene silencing efficiency was observed for THCO (61.1±2.2% to 46.3±4.8%), Transfast (62.3±1.6% to 41.8±8.2%) and DOTAP (47.1±3.8% to 21.9═7.7%) in the presence of 10% FBS as compared to that in serum free medium (FIG. 12). The pegylated THCO/siRNA nanoparticles showed serum-friendly transfection efficiency. Not wishing to be bound by theory, the pegylation at the surface stabilized the nanoparticles and protected siRNA against enzymatic degradation in the lysosomal compartments.

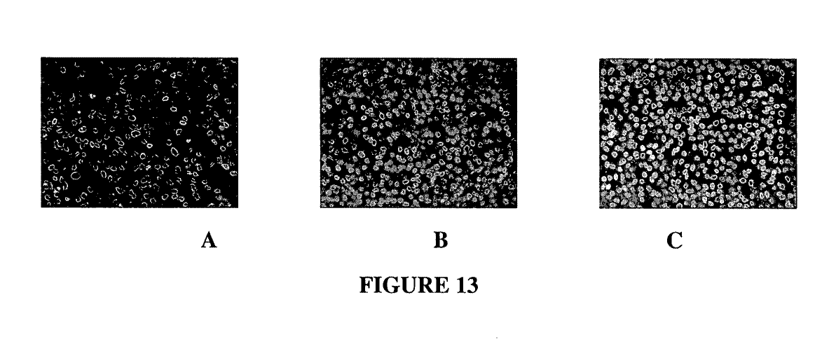

MFC mediated delivery of siRNA targeting endogenous housekeeping gene (Lamin A/C). Cells were plated into 8-well chamber microscope slides and incubated multifunctional carrier/siRNA complexes for 4 h at 37° C. in a 5% CO₂ environment. After incubation, the cells were washed and supplemented with fresh medium and allowed to incubate for an additional 44 h for RNAi to take effect. For fluorescence microscopy analysis, cells were washed by PBS containing 1% bovine serum albumin and then fixed with methanol on chambered slides. After cell fixation, cells were incubated with primary antibody to lamin (Abcam) and then with a fluorescently labeled secondary antibody (Aldrich). Gene silencing efficiency was visualized by fluorescence microscopy (FIG. 13). The cells treated with the THCO/siRNA and Transfast/siRNA complexes exhibited much weaker fluorescence than the untreated cells due to silencing of the expression of lamin A/C protein by RNAi.

Quantitative evaluation of MFC-mediated delivery of siRNA targeting firefly luciferase. The human astrocytoma cell line U373 MG with constitutive firefly luciferase expression (U373-Luc) was generated by infecting U373 MG cells with recombinant retroviruses containing a luciferase gene. U373-Luc cells were maintained in minimal essential medium (ATCC) containing 10% fetal bovine serum, G418 (300 μg/mL), streptomycin (100 μg/mL) and penicillin (100 units/mL).

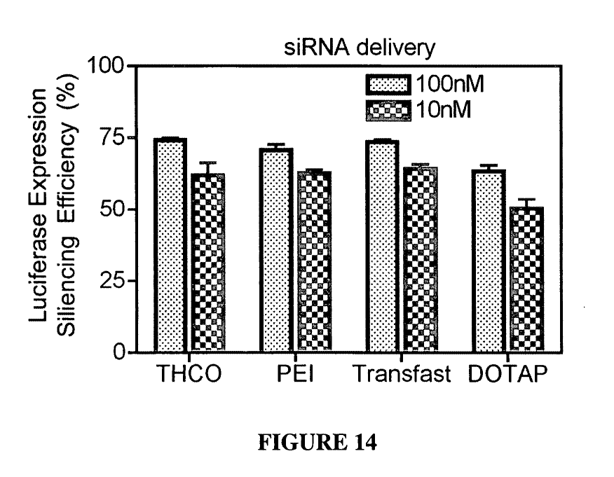

U373 MG-Luc cells were seeded 24 hours prior to transfection into a 96-well plate at a density of 2000 cells/well. At the time of siRNA transfection, the medium in each well was replaced with fresh serum-free medium. The complexes of carrier/anti-luciferase siRNA complexes were incubated with the cells for 4 hours at 37° C. The medium was then replaced with 1 mL of fresh complete medium and cells were incubated for an additional 44 hours. All the transfection tests were performed in triplicate wells. Cells were pelleted and then resuspended in 100 μL lysis buffer (Promega), and subjected to two cycles of freezing and thawing. Cellular debris was sedimented at 14,000 g for 1 minute, and 20 μL of the supernatant was assayed using the luciferase assay system (Promega). FIG. 14 shows luciferase gene silencing using THCO, PEI, Transfast, and DOTAP as the transfection agent. THCO demonstrated versatile high delivery efficiency regardless of the difference of the nucleic acids (PDNA or siRNA), cell lines (HeLa or MDA-MB-231), plasmids (reporter gene encoding either firefly luciferase or GFP) and siRNAs (targeting either endogenous housekeeping gene encoding Lamin A/C or recombinant gene encoding firefly luciferase).

E. Synthesis and Purification of Additional Monomeric Multifunctional Carrier Compounds

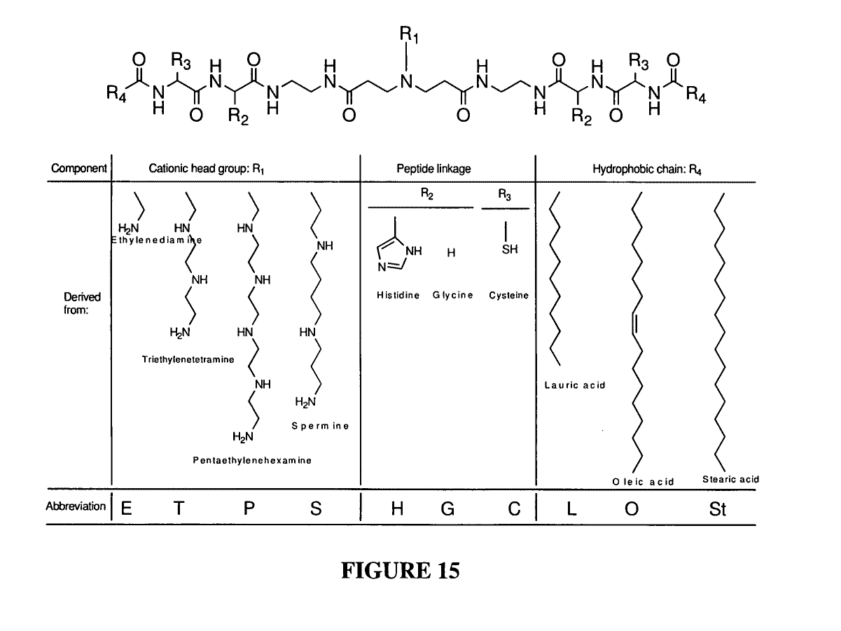

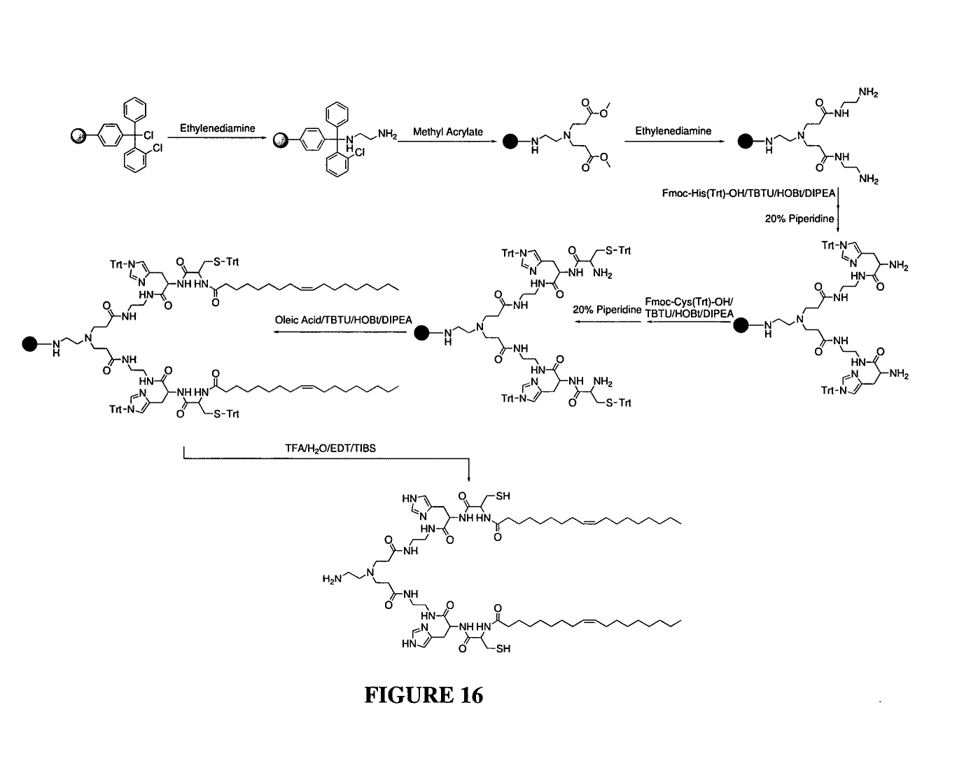

The MFCs were synthesized by solid phase chemistry. The synthetic procedure of (1-aminoethyl)imino-bis[N-(oleicyl-cysteinyl-histinyl-1-aminoethyl)propion-amide] (EHCO) is described as the representative procedure for the synthesis of the library of compounds. The synthetic protocol is presented in FIG. 16. The general structure of the MFCs is shown in FIG. 15, and specific compounds are shown in FIG. 2.

2-Chlorotrityl chloride resin (300 mg) was extensively washed with anhydrous DCM. A mixture of ethylenediamine (1.0 mL, excess) and DIPEA (64 mg) in DCM was added to the resin, and the suspension was shaken for 2 h. The solvent was drained and the resin was washed with DCM and MeOH. The resin was further shaken with 10 mL DCM/MeOH/DIPEA (17/2/1, v/v/v) for 20 min. The resin was then mixed with methyl acrylate (50 mL, excess) in 10 mL DMF to introduce methyl carboxylate via Michael addition. The reaction was carried out in a rotary evaporator at 50° C. with continuous rotating. A solution of 1,2-ethylenediamine (50 mL, excess) in 10 mL DMF was then mixed with the resin methyl carboxylate. The mixture was rotated in a rotary evaporator at 50° C. for 5 days. The resin containing primary amines was transferred into an ISOLUTE column, mixed with a solution of activated N-α-Fmoc-N-im-trityl-L-histidine (2.0 g, excess) with TBTU/HOBt/DIPEA (excess) in DMF and shaken for 2 hours. The resin was subjected to a washing cycle and Fmoc protecting group was removed with 20% piperidine in DMF (20 min×3) to give the resin containing histidine residues. Cysteine residues were similarly incorporated by reacting the resin with N-α-Fmoc-S-trityl-L-cycteine (2.0 g, excess) and TBTU/HOBt/DIPEA (excess) in DMF, followed by removal of Fmoc protecting groups. Finally, oleicyl groups were incorporated by reacting the resin with oleic acid (2 g) in the presence of TBTU/HOBt/DIPEA in DMF for 2 h. The quality of each coupling reaction involving primary amino groups was monitored with Kaiser test. The resin in each reaction cycle was extensively washed with DMF, MeOH and DCM, and dried under reduced pressure before proceeding to next reaction. The final resin was then suspended in a solution of TFA/H₂O/EDT/TIBS (94/2.5/2.5/1) and shaken for 3 h at room temperature. The solution was collected and concentrated under reduced pressure. The residue was washed with cold diethyl ether (40 mL×5) and dried. Final product EHCO was purified by preparative HPLC equipped with ZORBAX PrepHT C-18 column using an Agilent 1100 series purification system. Product fractions were collected and lyophilized. The purity of the final product was verified by analytic HPLC. The structure of the compound was analyzed by ¹H NMR spectroscopy using a Varian Mercury 400 (Palo Alto, Calif.) and matrix-assisted laser desorption ionization (MALDI) time-of-flight (TOF) mass spectrometry.

F. Physiochemical and Biological Properties of MFC and MFC/Nucleic Acid Complexes

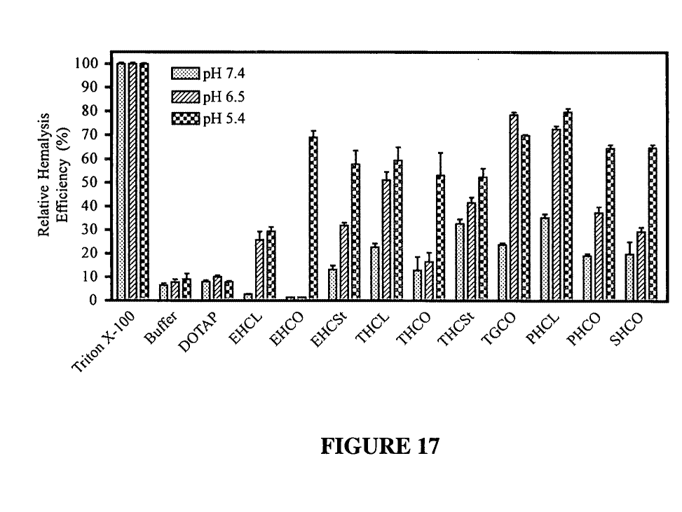

Hemolysis assay. The assays were performed using the techniques described above. The pH sensitive amphiphilic cell membrane disruption of the MFCs was evaluated by hemolysis assay with rat red blood cells at different pHs. FIG. 17 shows the hemolytic activity of the compounds at pH 7.4, 6.5 and 5.4 in PBS buffer. The positive control Triton X-100 (1%, w/v) resulted in complete hemolysis. Little hemolysis was shown with the buffer and DOTAP in all cases. The MFCs showed various pH dependent hemolytic activities. Generally, all MFCs had lower hemolytic activity at pH 7.4 than at pH 6.5 and 5.4. At pH 7.4, EHCL and EHCO resulted in negligible hemolysis, while the rest of the MFCs showed moderate hemolytic activities with 10-35% hemolysis. At pH 6.5, hemolytic activity of the MFCs increased except for EHCO, which still showed negligible hemolysis. At pH 5.4, all MFCs exhibited high hemolysis, and the MFCs except EHCL resulted in 50-80% hemolysis.

The pH sensitive hemolysis of the MFCs is governed by their structural characteristics. For the MFCs containing histidine residues and the same lipophilic tails, the hemolytic activity increased with increasing number of protonatable amino groups in the head group at pH 7.4 and 6.5. More protonatable amino groups could result in more charges in the head groups and higher amphiphilicity for the MFCs at neutral pH. For the EHC and THC series, the unsaturated oleicyl group resulted in less hemolysis than the saturated lipophilic tails in the same series at pH 7.4 and 6.5 except for EHTL at pH 7.4. The comparison between THCO and TGCO suggests that the histidine residues are important for pH sensitive amphiphilicity of the MFCs at endosomal-lysosomal pH. Overall, the pH sensitive hemolysis, disruption of membrane of red blood cells, is caused by the pH sensitive amphiphilicity of the MFCs derived from the combination of protonatable amino head groups and lipophilic tails. When more amino groups are protonated, the MFCs become more amphiphilic and result in more significant hemolysis. The protonation and pH sensitive amphiphilicity may be governed by the overall pKa of the head groups of the MFCs.

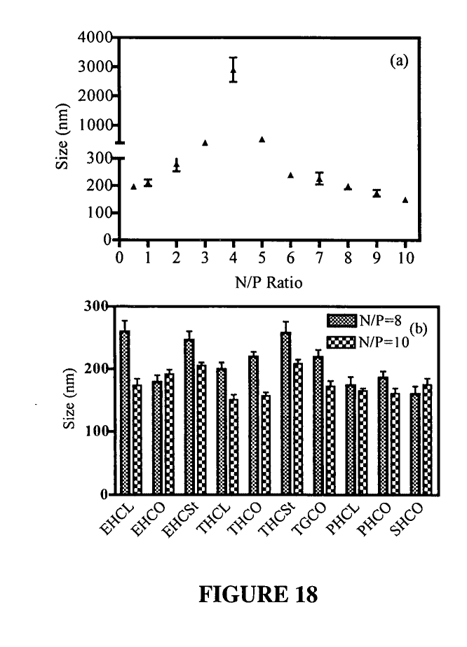

Formation and size measurement of MFC/siRNA nanoparticles. MFC/siRNA nanoparticulate complexes were prepared and characterized using the techniques described above. The complexation of the polymerizable MFCs with siRNA and formation of nanoparticles of the complexes were investigated by dynamic light scattering. EHCO was first used to study the complexation with siRNA and the impact of N/P ratios on the complexation and formation of nanoparticles. No nanoparticulate formation was detected in either EHCO or siRNA solutions. When EHCO was mixed with siRNA and incubated for 30 minutes, the formation of nanoparticulate complexes was observed with an N/P ratio as low as 0.5. The particle size changed with N/P ratios of the complexes (FIG. 18A). The initial size was approximately 200 nm in diameter at an N/P ratio of 0.5, and the size then increased with increasing N/P ratio up to an N/P ratio of 4. The particle size was as large as about 3 μm at the N/P ratio of 4, possible due to aggregation of relatively neutral complex particles. The particle size decreased to approximately 240, 200 and 151 nm at the N/P ratios of 6, 8 and 10, respectively. The particle size of siRNA complexes with other polymerizable surfactants was measured at the N/P ratios of 8 and 10 based on the observation with EHCO. As shown in FIG. 18B, the average particle sizes of the complexes were in the range of 160 to 260 nm at the N/P ratio of 8, and 160 to 210 nm at the N/P ratio of 10. The sizes of most complexes were under the reported cut-off size of 250 nm for efficient cellular uptake.

Autoxidation of the MFCs. The MFCs were diluted from stock solutions (N₂-protected, 2 mg/mL) to an initial thiol concentration of 150 μM in Tris buffer (10 mM pH 8.0). The autoxidation of the dithiols in the surfactants was performed with the working solutions (400 μL) in the absence or presence of 10 μg siRNA (N/P=10). For each predetermined time point, an aliquot was taken and mixed with equal volume of Ellman's solution (2 mM DTNB in 50 mM NaOAc solution), and the free thiol concentration was determined by Ellman assay using UV-Vis spectrophotometry (Cary-300 Bio).

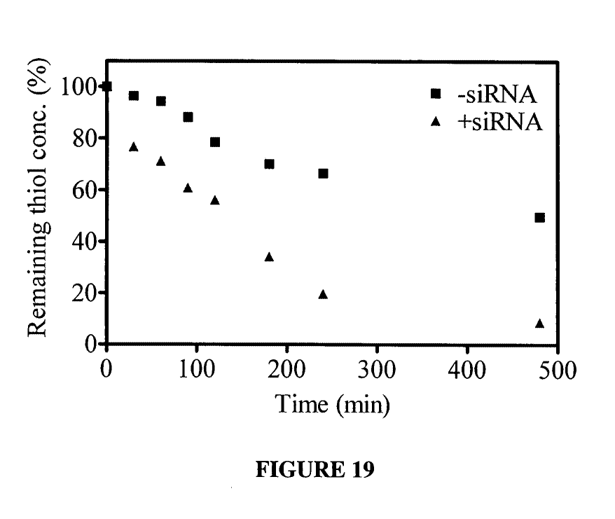

The MFCs formed relatively compact and small nanoparticles as compared to other reported siRNA delivery systems. Not wishing to be bound by theory, it is believed that complexes were formed by charge interaction between the carriers and siRNA. Hydrophobic interaction of lipophilic tails and polymerization of dithiols facilitated the formation of stable compact nanoparticles. The polymerization of the MFCs via oxidation was confirmed by the disappearance of thiols after they were mixed with siRNA, as determined by Ellman's assay (FIG. 19). The autoxidation rate of thiols was faster in the presence of siRNA than that in the absence of siRNA, possibly because the complexation with siRNA facilitated autoxidation. These disulfide-linkages could further stabilize the nanopaticulate complexes. Due to the significant difference of thiol/disulfide redox potential between the extracellular space and intracellular environment, the disulfide bonds would be stable in the extracellular space during the delivery process and then reduced in cytosol, facilitating the disassociation of nanoparticles and release of siRNA.

Cytotoxicity of MFC/siRNA complexes. U87-luc cells were incubated with MFC/siRNA complexes of different carriers according to the protocol described above. MTT in PBS (5 mg/mL, 25 μL) was added to each well after incubation and cells were incubated for another 2 hours. After cell culture medium was removed, 200 μL DMSO was added to each well and cells were incubated at 37° C. for 5 min. The optical absorption was measured at 570 nm using a micro-plate reader (Model 550, Bio-Rad Lab. Hercules, Calif.). The relative cell viability was calculated with the equation ([Abs]ₛₐₘₚₗₑ-[Abs]_{blank})/([Abs]_{control}-[Abs]_{blank})×100%.

G. In vitro delivery of nucleic acids by MFCs MFC-mediated in vitro gene silencing with siRNA. U87-luc cells with constitutive expression of firefly luciferase were maintained in minimal essential medium (ATCC) containing 10% FBS, G418 (300 μg/mL), streptomycin (100 μg/mL) and penicillin (100 units/mL). U87-Luc cells were seeded 24 hours prior to transfection into a 96-well plate at a density of 5000 cells/well. At the time of siRNA transfection, the medium in each well was replaced with fresh serum-free medium. Anti-luciferase siRNA was complexed with the MFC, TransFast or DOTAP and incubated for 30 min before use. Complexes were incubated with the cells for 4 hours at 37° C. The medium was then replaced with 100 μL of fresh complete medium and cells were incubated for an additional 44 hours. The cells were then washed with pre-warmed PBS, treated with 200 μL cell lysis buffer and subjected to a freezing-thawing cycle. Cellular debris was removed by centrifugation at 14,000 g for 5 minutes. The luciferase activity in cell lysate (20 μL) was measured using a luciferase assay kit (100 μL luciferase assay buffer) on a luminometer for 10 seconds (Lumat 9605, EG&G Wallac). The gene silencing efficiency was normalized against the luciferase expression of untreated cells.

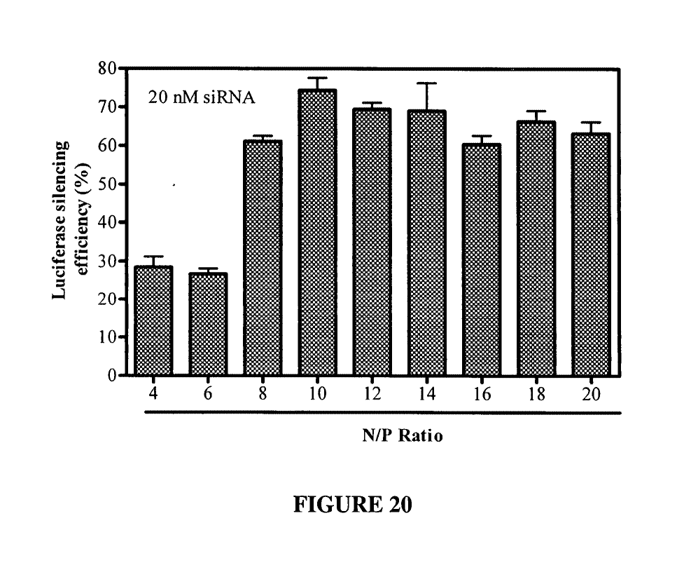

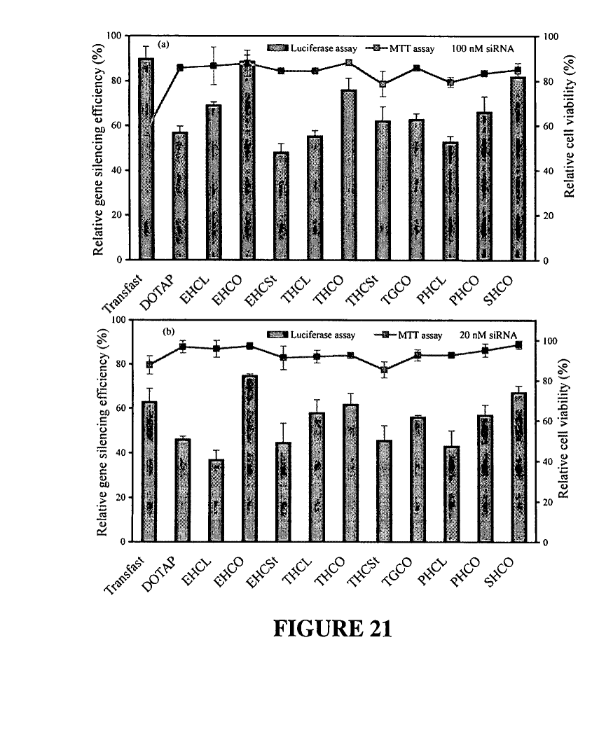

The efficacy of the MFCs for cellular siRNA delivery was evaluated using an anti-luciferase siRNA in U87-luc cell line with stable expression of firefly luciferase. Commercial transfection agents TransFast and DOTAP were used as controls. Silencing efficiency of luciferase expression mediated by the surfactants was first evaluated with EHCO at different N/P ratios to obtain the best N/P ratios for effective siRNA delivery. High luciferase expression knockdown efficiency was observed with N/P ratios ranging from 8 to 20 for EHCO (FIG. 20). The peak silencing efficiency was observed at the N/P ratio of 10 for the MFCs. The silencing efficiency of luciferase expression mediated by the MFCs was evaluated at the fixed N/P ratio of 10. Viability of cells incubated with the siRNA complexes was also investigated at the same time by MTT assay to assess the cytotoxicity of the MFCs. As shown in FIG. 21a, TransFast resulted in 89.6±5.6% knockdown of luciferase expression as compared to untreated cells with 100 nM siRNA, but significant cytotoxicity was observed with cell viability of only 57.6±2.2%. The relatively high gene silencing efficiency of TransFast with 100 nM siRNA could also be the result of low cell viability. DOTAP/siRNA complexes at the same siRNA concentration had higher viability (85.8±1.6%), but resulted in significantly lower luciferase knockdown efficiency (56.7±3.1%). The cells incubated with the siRNA complexes of the MFCs had a relatively high viability ranging from 78.6±5.7% to 88.2±1.3%. The luciferase expression knockdown efficiencies varied from 47.8±4.2% to 88.4±3.1%. EHCO resulted in the highest gene silencing efficiency (88.4±3.1%) with high cell viability (86.7±8.3%) among the MFCs.All of siRNA complexes demonstrated low cytotoxicity at a low siRNA concentration (20 nM) (FIG. 21b). Cell viability was high in all cases including Transfast (87.6±4.6%). Under this condition, the MFCs except EHCL resulted in higher luciferase silencing efficiency than DOTAP. THCL, THCO, TGCO, PHCO and SHCO resulted in comparable transfection efficiency to TransFast (62.6±6.4%). EHCO (74.5±1.0%) resulted in significantly higher transfection efficiency than TransFast. The cellular siRNA delivery efficiency correlated well to the pH sensitive hemolytic activity and amphiphilicity of the MFCs. EHCO exhibited the highest gene silencing efficiency at both siRNA concentrations. THCO and SHCO with relatively low hemolysis at pH 7.4 and 6.5 also showed relatively high gene silencing efficiency among the MFCs.

H. In Vivo Gene Silencing with EHCO

Animal tumor model. U87 cells with stably expression of luciferase were collected and resuspended with medium/matrigel mixture (v/v=l/l). 100 μL of cell suspension containing 2 million cells were injected subcutaneously in the right flanks of the mice.

Carriers. EHCO was selected for the study. DOTAP is a commercial available transfection agent that has been used to deliver siRNA in vivo and was used as a control carrier. siRNAs. Anti-Luciferase siRNA was used to knockdown luciferase expression in mice so as to noninvasively detect gene silencing efficiency by bioluminescence imaging. Hypoxia-inducible factor-1a (Hif) is related to regulate vascular endothelial growth factor (VEGF) expression, and intratumor injection of anti-Hif siRNA complexes has been demonstrated to inhibit tumor growth in vivo. siRNA targeting firefly luciferase was purchased from Dharmacon (Chicago, Ill.). The sequence of antisense is 5′-UCGAAGUACUCAGCGUAAGdTdT-3′ and that of sense is 3′-dTdTAGCUUCAUGAGUCGCAUUC-5′. siRNA targeting HIF was provided Huntsman Cancer Institute, University of Utah. The sequence of antisense is 5′-UCACCAAAGUUGAAUCAGAdTdT-3′ and that of sense is 3′-dTdT AGUGGUUUCAACUUAGUCU-5′.

Administration of Anti-Luc siRNA complexes and Luciferase mouse imaging. Bioluminescence imaging of mice were taken one day before the administration of the anti-luciferase complexes. Mice were injected i.p. with 50 mg/kg firefly D-Luciferin (Xenogen Corp., Alameda, Calif.), 100 mg/kg ketamine, and 10 mg/kg xylazine hydrochloride (both from Sigma) in PBS. Mice were photographed 10 min after injection with an IVIS 100 imaging system (Xenogen) with an exposure time of 1 sec, medium binning. Images were quantified using LivingImage software (Xenogen). On the following day, 50 μg anti-luciferase siRNA in 300 μL was mixed with EHCO or DOTAP and the resulting complexes were injected into mouse via the systemic intraperitoneal injection. Bioluminescence images of the mice were taken two days after injection of the siRNA complexes. The luminescence signal intensity on the day before the mice received the anti-luc complexes were set as 100% and the subsequent luciferase expression level in mice after siRNA treatments were normalized to the percentage relative to those at day 0.

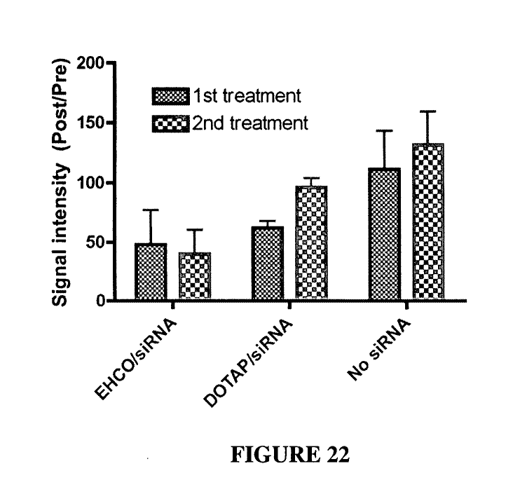

FIG. 22 shows that EHCO resulted in more than 50% reduction of luciferase expression in the tumor after two treatments, while DOTAP only had an initial effect after the first treatment and did not have significant effect after the second treatment.

Administration of Anti-Hif siRNA complexes and tumor size measurements. Anti-Hif siRNA complexes were injected on day 21 after tumor cell inoculation. A group of mice (n=6) were injected siRNA complexes by I.P. injection twice a week for the first 3 weeks and once a week for the fourth week. The administration of the siRNA complexes was repeated seven 7 times in total. The other group (n=8) was used as a control. The growth of the tumors was monitored every 2-4 days by measuring the tumor volume with a digital caliper. Tumor growth was measured using a digital caliper. Tumor volume was calculated by the formula 0.52× longest diameter×shortest diameter². The day that mice received anti-Hif siRNA complexes was set as day 0, and the tumor volume was normalized to 100%. All subsequent tumor volumes were also expressed as the percentage relative to those at day 0.

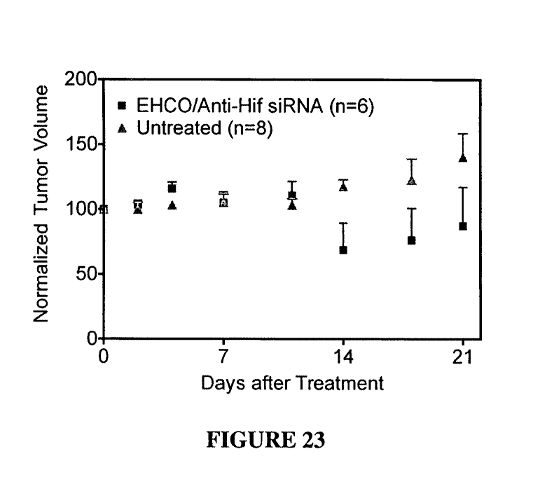

EHCO mediated high anti-tumor efficacy with an anti-HIF siRNA upon systemic i.p. administration in the subcutaneous mouse tumor model with U87-luc glioma xenograft in a preliminary study. Five out of six mice injected with the EHCO/anti-HIF siRNA nanoparticles at a siRNA dose as low as 2 mg/kg showed significant response to the treatment after 27 days. FIG. 23 shows relative tumor volume changes of the mice responded to the treatment and those of untreated mice during the period of the treatment. The tumor growth curve of the non-responding mouse in the treatment group is also shown in FIG. 23.

II. Disulfide Multifunctional Compounds and Characterization thereof

A. General

2-Chlorotrityl chloride resin (1.1 mmol/g), N-fluorenylmethoxycarbonyl-N-im-trityl-L-histidine (Fmoc-His(Trt)-OH), N-fluorenylmethoxycarbonyl-S-trityl-L-cysteine (Fmoc-Cys(Trt)-OH), 2-acetyldimedone (Dde-OH), 2-(1H)-benzotriazole-1-yl)-1,1,3,3-tetramethyluronium tetrafluoroborate (TBTU) and N-hydroxybenzotriazole (HOBt) were purchased from EMD Biosciences (San Diego, Calif.). Triethylenetetramine, N,N-diisopropylethyleneamine (DIPEA), methyl acrylate, 1,2-ethylenediamine, hydrazine, 4-dithio-DL-threitol (DTT), triisobutylsilane (TIBS), 1,2-ethanedithiol (EDT), piperidine, trifluoroacetic acid (TFA) were purchased from Lancaster (Windham, N.H.). Hyperbranched PEI (Mw=25 KDa), heparin sulfate, chloroquine diphosphate (CQ) and 2,5-diphenyl-3-(4,5-dimethyl-2-thiazolyl)tetrazolium bromide (MTT) were purchased from Sigma-Aldrich (St. Louis, Mo., USA). Dimethyl sulfoxide (DMSO), N,N-dimethylformamide (DMF) and dimethanechloride (DCM) were extra-dry solvents and they were purchased from Acros (Pittsburgh, Pa.). For solid-phase synthesis, except other mentioned, the reactions were performed in ISOLUTE column reservoirs equipped with caps and frits (Charlottesville, Va.). gWiz™ reporter plasmid encoding luciferase was purchased from Aldevron (Fargo, N. Dak.). siRNA targeting Firefly Luciferase was purchased from Dharmacon (Chicago, Ill.). The sequence of antisense is 5′-UCGAAGUACUCAGCG UAAGdTdT-3′ and that of sense is 3′-dTdTAGCUUCAUGAGUCGCAUUC-5′. Triethylenetetramine was purified by distillation under reduced pressure. All other materials and solvents were used without additional purification.

B. Synthetic Procedure

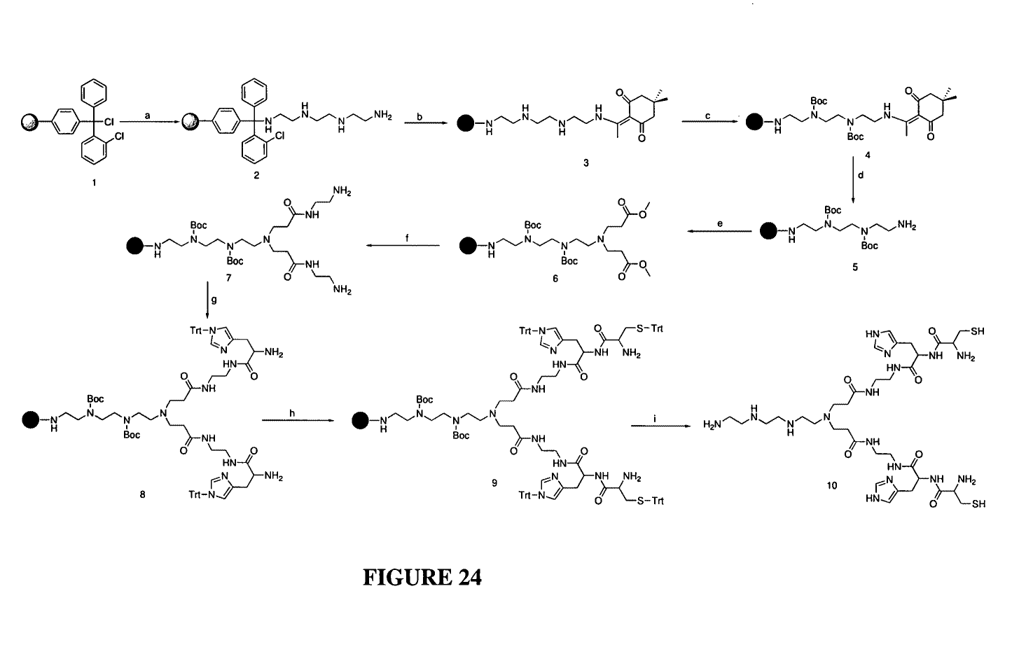

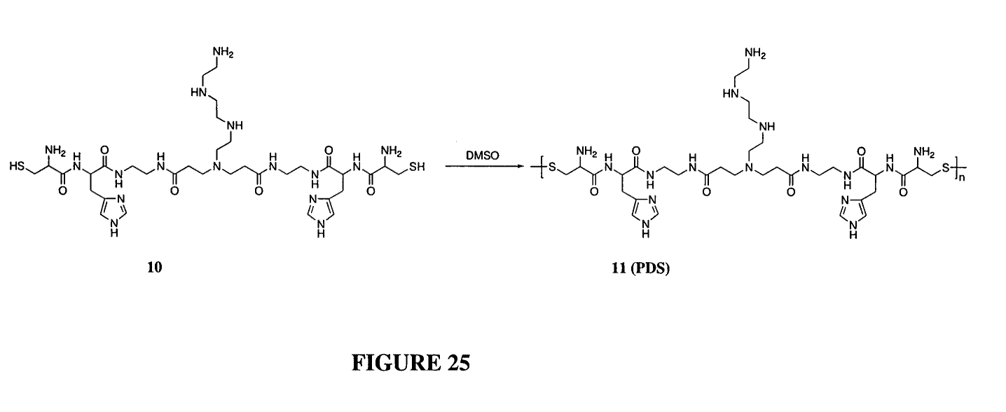

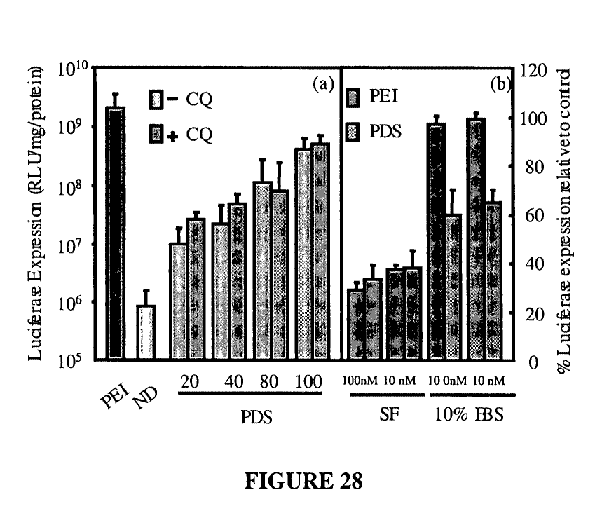

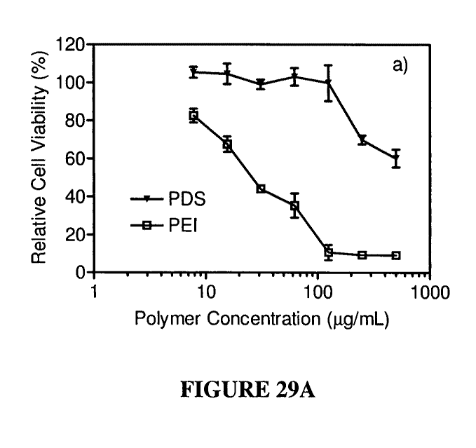

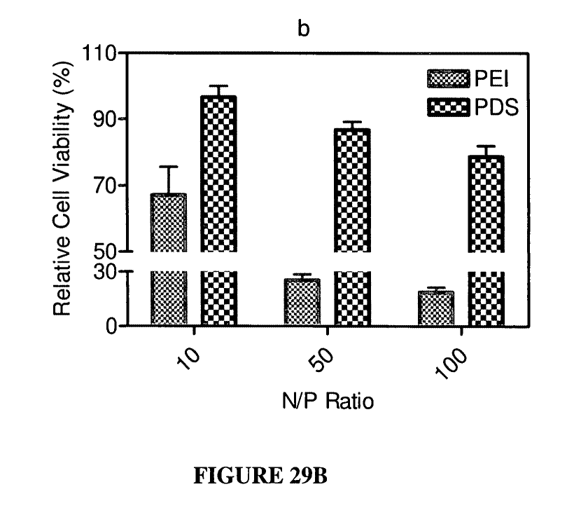

FIG. 24 shows the resin-supported synthesis of dithiol-containing monomer possessing primary, secondary and tertiary charge groups. FIG. 25 shows the synthesis of polydisulfide oligomer by oxidative polymerization. Reference numbers below are the same as those in FIGS. 28 and 29.

1-[2-chlorotrityl-amino]-1,4,7,10-tetraazadecane resin (Resin 2). 2-chlorotrityl chloride resin (300 mg, 1.1 mmol/g) was poured into the reaction vessel and extensively washed with dry DCM. A solution of triethylenetetramine (1 mL) and DIPEA (64 mg) in DCM was added to the resin, and the suspension was shaken for 2 h. The solvent was drained, and then washed with DCM and MeOH. The resin was further shaken with 10 mL DCM/MeOH/DIPEA (17/2/1, v/v/v) for 20 min. The resulting resin 2 was extensively washed with DMF, DCM and dried under reduced pressure.

Selective protection and deprotection of polyamine with 2-acetyldimedone (Dde-OH) and tert-butyloxycarbonyl (Boc) groups. A solution of 2-acetyldimedone (Dde-OH, 2 g) in 10 mL DMF was added to resin 2. The suspension was shaken at room temperature for 12 h. The resin was extensively washed with DMF and DCM, and solvent was drained to give resin 3. The resin 3 was suspended in the solution of Boc₂O (10 g) in 15 mL DCM. The mixture was shaken at room temperature for 4 h. The resulted resin was extensively washed with DMF and DCM, dried under reduced pressure to afford resin 4. Next, the resin 4 was suspended in the solution of 2% hydrazine in DMF for 15 min. This step was repeated three times to ensure complete deprotection of Dde groups. The resulted resin was extensively washed with DMF and DCM to give resin 5. It was dried under reduced pressure for the next step.

Elongation sequentially with methyl acrylate, 1,2-ethylenediamine, N-α-Fmoc-N-im-trityl-L-histidine and N-α-Fmoc-S-trityl-L-cycteine. Dry resin 5 was transferred into 100 mL round flask and methyl acrylate (50 mL) and DMF (10 mL) were added. The reaction was carried out in a rotary evaporator at 50° C. with continuous shaking. The reaction was monitored by Kaiser ninhydrin test until the primary amine was completely consumed. Resin 6 was extensively washed with DMF, MeOH, DCM and dried under reduced pressure.

For the next step, a solution of 1,2-ethylenediamine (50 mL) in DMF (10 mL) was added to the resin 6. The reaction has been performed in a rotary evaporator at 50° C. with continuous shaking for 5 days. The resin was extensively washed with DMF, MeOH, DCM and dried under reduced pressure to give resin 7.

The resin was transferred back to an ISOLUTE column reservoirs equipped with caps and frits for next solid-phase reactions. A solution of activated N-α-Fmoc-N-im-trityl-L-histidine (2 g) with TBTU/HOBt/DIPEA in DMF was added to resin 7 and the coupling reaction was continued for 2 h. The quality of coupling was followed by Kaiser test. The resin was subjected to washing cycle and Fmoc protecting groups were removed by suspension resin in 20% piperidine in DMF (20 min×3) to afford resin 8. The resin was then extensively washed with DMF and DCM, dried under reduced pressure for the next step.

Subsequently, a solution of activated N-α-Fmoc-S-trityl-L-cycteine (2 g) with TBTU/HOBt/DIPEA in DMF was added to resin 8 and the coupling reaction was continued for 2 h. The quality of coupling was followed by Kaiser test. The resin was extensively washed and Fmoc protecting groups were removed with 20% piperidine in DMF. The resin 9 was then extensively washed with DMF, MeOH and DCM for the next step.

Cleavage and Deprotection of Monomer from the Resin. The resin 9 was suspended in a solution of TFA/H₂O/EDT/TIBS (94/2.5/2.5/1). After shaking for 3 hours at room temperature, the solution was collected and concentrated under reduced pressure. The residue was washed with cold diethyl ether (40 mL×5) and dried. Monomer 10 was further purified by preparative HPLC to yield 60 mg product. ¹H NMR (400 MHz, D₂O) δ δ 8.45 (m, 2H), 7.2 (m, 2H), 4.5 (m, 2H), 4.05 (m, 2H), 3.2-2.8 (m, 24H), 2.7-2.5 (m, 8H), 2.4-2.3 (m, 4H) MS m/z calcd for C₃₄H₆₂N₁₆O₆S₂(M+H)⁺ 855.45, found 855.50

Oxidative Polymerization in DMSO. The oxidative polymerization was performed with 36 mg of monomer 10 in 100 μL of DMSO at 37° C. At certain time points during the oxidative polymerization, an aliquot of the reaction mixture was taken to detect molecular weight by FPLC. The polymerization was performed for 5 days. The polymer residue was obtained by precipitation with acetone (10 mL) and it was further purified by dialysis against ultra-pure water (MWCO=2000) overnight. Final product was dried by lyophilization. ¹H NMR (400 MHz, D₂O) δ8.34 (m, 2H), 7.12 (m, 2H), 4.5 (m, 2H), 4.1 (m, 2H), 3.2-2.8 (m, 24H), 2.7-2.5 (m, 8H), 2.4-2.3 (m, 4H) Mw=6.2K (determined by SEC).

C. Characterization of Polydisulfide and Polydisulfide/DNA Complexes

Gel Electrophoresis Shift Assay. The ability of polydisulfide 11 (PDS) to bind DNA was examined by gel electrophoresis. Agarose gel (0.8%, w/v) containing 0.5 μg/mL ethidium bromide was prepared in TAE (Tris-Acetate-EDTA) buffer. DNA (10 μL, 0.1 μg/μL) was mixed with an equal volume of polymer solution at predetermined N/P ratios (N=polymer nitrogens that can be protonated, P=phosphate groups on DNA) and incubated for 30 min before use. 10 μL of each sample was mixed with 2 μL of 6× loading dye and the mixture was loaded onto an agarose gel. The gel was run at 100V for 60 min. The location of DNA bands was visualized on a UV illuminator.

The ability of reducing conditions to destabilize PDS/DNA polyplexes was examined by incubating polyplexes (N/P=3) with 0.1 M DTT at 37° C. for 4 h in the presence/absence of 0.15M NaCl or 1 mg/mL heparin. The samples were then analyzed by gel electrophoresis as described above.

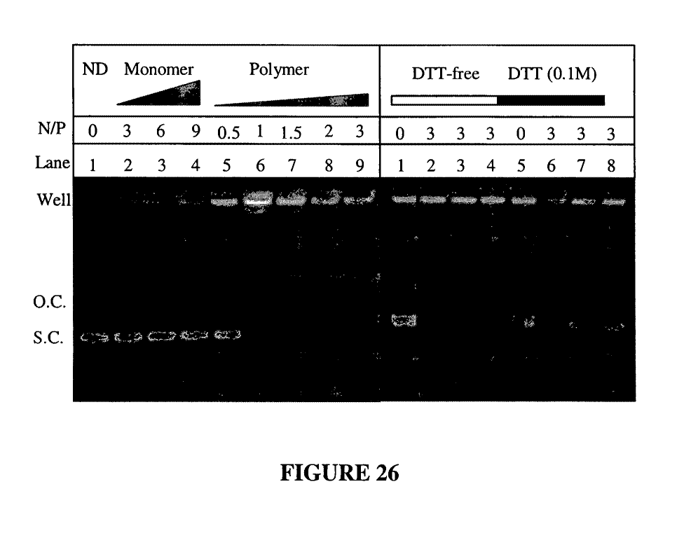

FIG. 26 shows the gel electrophoresis shift assay at the indicated N/P ratios. Lane 1: DNA only; Lane 2-4: monomer/DNA complexes; Lane 5-9: polymer/DNA complexes. (b) Reduction destabilizes polyplexes in the presence of NaCl or heparin. Polyplexes with N/P ratio of 3 were used for the study. Lane 1-4 were performed in the absence of DTT, lane 5-8 were performed in the presence of 0.1 M DTT. Polyplexes were incubated for 4 h at 37° C. Lane 1: DNA only; Lane 2: polyplexes only; Lane 3: polyplexes with 0.15 M NaCl; Lane 4: polyplexes with 0.1 mg/mL Heparin. Lane 5: DNA only; Lane 6: polyplexes only; Lane 7: polyplexes with 0.15 M NaCl; Lane 8: polyplexes with 0.1 mg/mL Heparin. (ND=naked DNA, O.C.=open circular, S.C.=supercoiled form of plasmid DNA).

As shown in FIG. 26(a), monomer cannot retard DNA migration in wells up to an N/P ratio of 9. In contrast, DNA was partially retained in the well at an N/P ratio of 11 by the polydisulfide and completely retained at an N/P ratio of 1.5 or higher. The polyplexes exhibited high stability at an N/P ratio of 3 in the presence of salt (0.15 M NaCl) or negatively charged heparin (1 mg/mL). As shown in FIG. 26(b), no detectable dissociation of the polyplexes was resulted from physiological ionic strength and competitive biomacromolecules. The release of DNA from the polyplexes was also investigated in a reductive environment under different conditions. The presence of disulfide reducing agent DTT led to the release of free DNA. High ionic strength or the presence of heparin further facilitated the release of DNA in the reductive environment. The results suggested that the DNA polyplexes with the cationic polydisulfide are stable extracellularly yet able to release DNA intracellularly to enhance delivery efficiency.

Particle Size Analysis. Samples were prepared by mixing 5.0 μg of plasmid DNA or siRNA with appropriate amount of materials in dust-free water and analyzed using a Brookhaven Instruments BI-200SM system equipped with a 5 mW helium neon laser with a wavelength output of 633 nm. The effective diameter and population distribution were computed from the diffusion coefficient using functions supplied by the instrument. Measurements were made at 25° C. at an angle of 90°. Each sample was analyzed in triplicate, and the reported data represent the mean values. FIG. 27 shows size of PDS/DNA and PDS/siRNA complexes at indicated N/P ratios.

Acid-base Titration Assay. Acid-base titrations were carried out by using a 6-mL solution of PDS or PEI (5 mM based on positive charge) and the initial pH was adjusted to 10. Sequential 2 μL of 1 M HCl were performed, and pH after each addition was measured; a 5 mM solution of NaCl was titrated similarly as a control. The PDS exhibited similar buffering capacity as PEI in endosomal-lysosomal pH range (4.5˜7.2) according to pH titration curve. This is due to the combination effect of the primary, secondary, tertiary and aromatic amino groups in the polymer.

D. Cell Culture Experiments

In Vitro protocol for plasmid DNA delivery. COS-7 (monkey SV40 transformed kidney fibroblast cells) and MDA-MB-231 (Human Caucasian breast adenocarcinoma epithelial cells) were obtained from ATCC (American Type Culture Collection, Rockville, Md., USA). Cos 7 cells were maintained in Dulbecco's Modified Eagle's Medium (DMEM) and MDA-MB-231 cells were maintained in ATCC Leibovitz's L-15 medium at 37° C. in a humidified 5% CO₂ atmosphere. Growth medium was supplemented with 10% fetal bovine serum (FBS, HyClone, Logan, Utah), streptomycin (100 μg/mL) and penicillin (100 units/mL). Cells were seeded 24 hours prior to transfection into a 24-well plate. At the time of transfection, the medium in each well was replaced with 1 mL of serum free medium with or without 100 μM chloroquine. The complexes of Polymer/DNA or PEI/DNA at different N/P ratios were incubated with the cells for 4 hours at 37° C. The medium was then replaced with 1 mL of fresh complete medium and cells were incubated for an additional 44 hours. All the transfection tests were performed in triplicate wells. After the incubation, cells were treated with 200 μL of cell lysis buffer (Promega Co., Madison, Wis.). The luciferase activity in cell extracts was measured using a luciferase assay kit (Promega Co., Madison, Wis.) on a luminometer for 10 seconds (Lumat 9605, EG&G Wallac). The relative light units (RLU) were normalized against protein concentration in the cell extracts, measured by a BCA protein assay kit (Pierce, Rockford, Ill.). Luciferase activity was expressed as relative light units (RLU/mg of protein in the cell lysate).

FIG. 28a shows transfection efficiency of PDS/DNA complexes in Cos 7 cells at different N/P ratios in the absence or presence of 100 μM CQ in comparison with PEI and Naked DNA. Mean±standard deviation (n=3). As shown in FIG. 28a, luciferase expression level mediated by the complexes was N/P ratio dependent. Polydisulfide mediated gene transfection peaked at N/P ratio of 100, and similar gene transfer capability was observed in MDA-MB-231 cells (Data not shown). A relative high charge ratio for optimal transfection was required for the cationic polydisulfide, which is similar to other reported biodegradable gene carriers. Nevertheless, PDS polyplexes (N/P=100) still are less toxic when compared to PEI (N/P=10). The transfection efficiency may correlate to the size of the polyplexes because the size decreased with the increase of N/P ratio, from a diameter of 320 at N/P=40 to 135 nm at N/P=80 (see supporting information). At optimal N/P ratio, the polydisulfide resulted in gene expression approximately 3-fold lower than PEI in Cos 7 cells. The presence of chloroquine diphosphate (CQ, 100 μmM), a reagent known to disrupt endosomal membrane, did not significantly improve gene expression mediated by polydisulfide, indicating that the cationic polydisulfide possessed high buffering capacity which may contribute to help polyplexes to escape from endosomal compartments.

In vitro protocol for siRNA delivery. The human astrocytoma cell line U373 MG with constitutive firefly luciferase expression (U373-Luc) (initial stock kindly provided by Dr. David Gillespie, Huntsman Cancer Institute, University of Utah) was generated by infecting U373 MG cells with recombinant retroviruses containing a luciferase gene. U373-Luc cells were maintained in minimal essential medium (ATCC) containing 10% fetal bovine serum, G418 (300 μg/mL), streptomycin (100 μg/mL) and penicillin (100 units/mL).

U373 MG-Luc cells were seeded 24 hours prior to transfection into a 96-well plate at a density of 2000 cells/well. At the time of siRNA transfection, the medium in each well was replaced with fresh serum-free or serum-containing medium. The complexes of carrier/anti-luciferase siRNA at different N/P ratios were incubated with the cells for 4 hours at 37° C. The medium was then replaced with 1 mL of fresh complete medium and cells were incubated for an additional 44 hours. All the transfection tests were performed in triplicate wells. Cells were pelleted and then resuspended in 100 μL lysis buffer (Promega), and subjected to two cycles of freezing and thawing. Cellular debris was sedimented at 14,000 g for 1 minute, and 20 μL of the supernatant was assayed using the luciferase assay system (Promega).

FIG. 28b shows endogenous luciferase gene silencing efficiency by PDS/siRNA or PEI/siRNA complexes in u373-Luc cells under different conditions. A and B were performed in serum-free condition at siRNA concentration of 100 and 10 nM, respectively. C and D were performed in the presence of 10% FBS at siRNA concentration of 100 and 10 nM, respectively. Mean±standard deviation (n=3). Luciferase silencing efficiency mediated by PDS/siRNA was as efficient as PEI/siRNA even at low siRNA concentration (10 nM). The luciferase expression was reduced to ˜30% for both polydisulfide/siRNA (N/P=30) and PEI/siRNA (N/P=10) complexes in a serum-free medium (FIG. 28b). Interestingly, PEI/siRNA complexes had no effect on gene silencing in the presence of 10% FBS, which is most likely due to unfavorable interaction between polyplexes and serum proteins. Under the same condition the polydisulfide/siRNA complexes showed serum-friendly that still exhibited up to 40% gene silencing efficiency.

Cytotoxicity assay. Cytotoxicity of polydisulfide in comparison with PEI was evaluated using MTT assay. MDA-MB-231 cells were seeded in a 96-well plate 24 h before the assay at a density of 10000 cells/well. The cells were incubated with 200 μL of complete L-15 medium containing polymers at different concentrations or polyplexes at different N/P ratios. After 4 h, the medium in each well was replaced with 100 μL of fresh complete medium. Then 25 μL MTT solution in PBS was added and cells were incubated for another 2 hours. After removing media, 200 μL of DMSO was added to the wells and cells were incubated at 37° C. for 5 min. The optical densities were measured at 570 nm using a microplate reader (Model 550, Bio-Rad Lab. Hercules, Calif.). The relative cell viability was calculated by ([Abs]ₛₐₘₚₗₑ-[Abs]_{blank})/([Abs]_{control}-[Abs]_{blank})×100%. FIG. 29a shows relative cell viability vs. PDS and PEI concentration, and FIG. 29b shows relative cell viability vs. N/P ratio of PDS and PEI. The cationic PDS exhibited much lower cytotoxicity than PEI.

III. Methods for MFC-Mediated Targeting delivery of Nucleic Acids

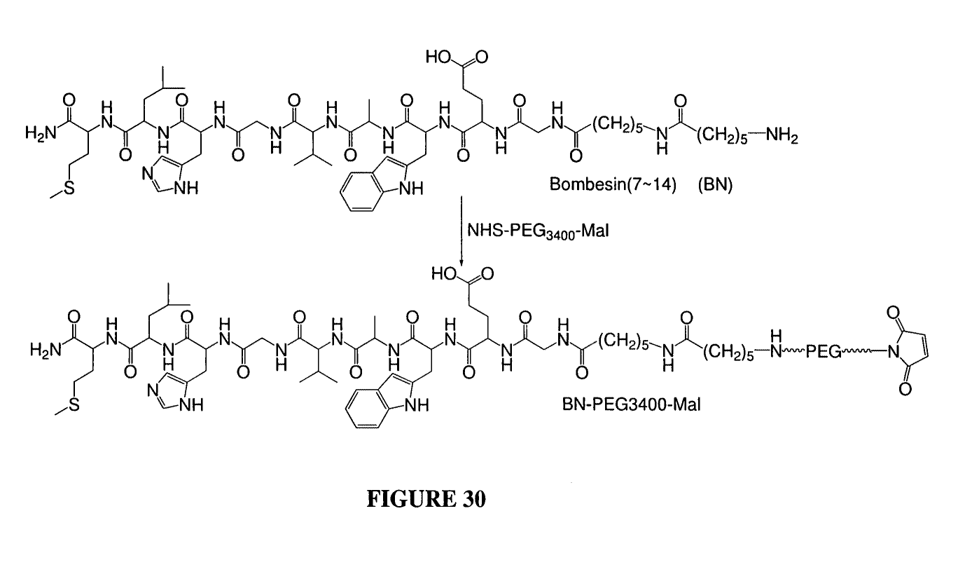

Synthesis of Bombesin-PEG-Mal conjugate. Bombesin (7-14) with two consecutive 6-amino-hexanoic acid units was synthesized on solid-phase supported resin. Bombesin peptide was purified by parepartive HPLC, lypholized and then reacted with NHS-PEG₃₄₀₀-Mal to obtain BN-PEG-Mal conjugate. The sythetic scheme is illustrated in FIG. 30. The molecular weight of BN-PEG₃₄₀₀-Mal was confirmed by MOLDI-TOF.

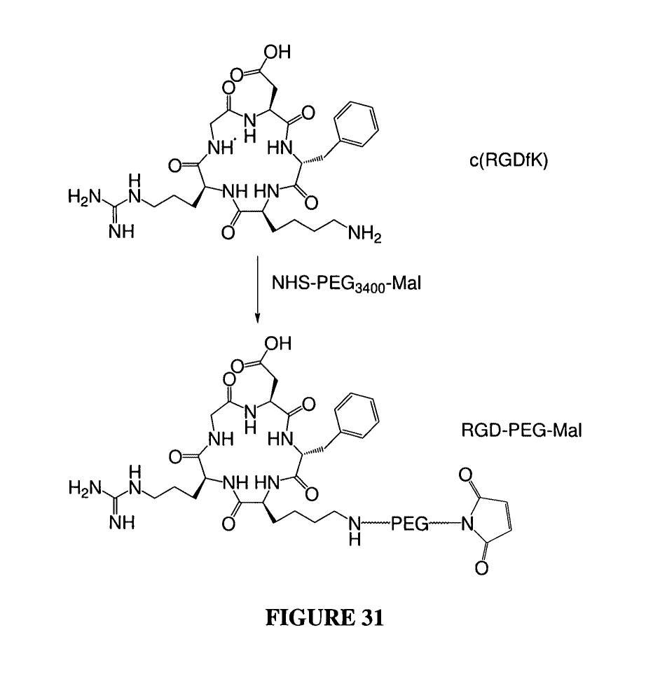

Synthesis of RGD-PEG-Mal conjugate. Cyclic peptide c(RGDfK) was purchased from Peptide International. It was reacted with NHS-PEG₃₄₀₀-Mal to obtain RGD-PEG-Mal conjugate. The sythetic scheme is illustrated in FIG. 31. The molecular weight of BN-PEG₃₄₀₀-Mal was confirmed by MOLDI-TOF.

General procedure for surface modification of MFC/siRNA or MFC/DNA nanoparticles. The surface modification of MFC/siRNA or MFC/DNA nanoparticles was performed by taking advantage of thiol-maleimide reaction. The general scheme to functionalize MFC/siRNA nanoparticles is illustrated in FIG. 32. For example, 1 μg siRNA was combined with 9 nmol of MFC-EHCO followed by adding the appropriate amount of maleimide-containing functional molecules (0.225 nmol of BN-PEG-Mal results in 2.5% BN-PEG-Mal modification degree based on BN/EHCO mole ratio). Nanoparticles could be modified by reaction with different maleimide-containing molecules, i.e. mPEG-Mal, BN-PEG-Mal or RGD-PEG-Mal. Similarly, fluorescein-labeled nanoparticles were obtained with fluorescein-5-maleimide. After 2 h incubation in the dark, free maleimide derivatives were removed by ultrafiltration (Nanosep®, MWCO=100K, 5000 g, 5 min) and the nanocomplexes were recovered for further experiments.

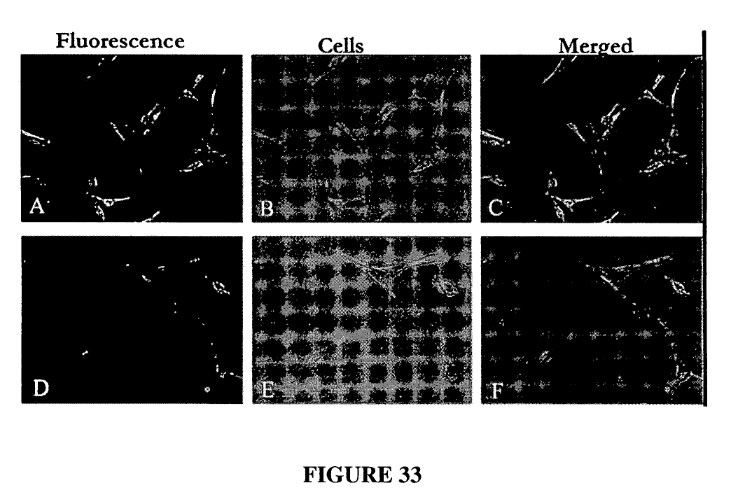

Fluorescence microscopy. Approximately 100,000 U87 cells were plated per well in a 12-well cell culture plate. Fluorescein-labeled MFC/DNA nanoparticles were prepared as described above. In the cases of complexes with ligands, appropriate amount of BN-PEG-Mal or RGD-PEG-Mal or mPEG-Mal were added in the complex mixtures. After 2 h incubation in dark, free maleimide derivatives were removed by ultrafiltration (Nanosep®, MWCO=100K, 5000 g, 5 min). The recovered samples were applied to cells and incubated at 37° C. for 2 h. The medium was removed by aspiration and the wells were washed with cold phosphate buffered saline (PBS) and fixed with 1 ml/well of 4% PFA in PBS at 4° C. for 20 min. The fixation agent was aspirated, and the cells were twice washed with PBS. The samples were visualized by fluorescence microscopy. The tumor specific peptide modified nanoparticles showed more significant uptake in cancer cells than the non-targeted nanoparticles (FIG. 33).

Cellular uptake of BN-PEG-Mal modified nanoparticles. Approximately 500,000 U87 cells were plated per well in a 6-well plate 24 hours prior to the study. Fluorescein-labeled EHCO/siRNA nanoparticles were prepared as described above. After EHCO/siRNA nanoparticles formation, an appropriate amount of BN-PEG-Mal or mPEG-Mal was added to the mixture. After 2 h incubation in the dark, free maleimide derivatives were removed by ultrafiltration (Nanosep®, MWCO=100K, 5000 g, 5 min). The recovered samples were applied to cells and incubated at 37° C. After 2 h the medium was removed by aspiration and the cells were washed twice with cold phosphate buffered saline (PBS) and then trypsinized. The cells were collected and fixed with 2% PFA in PBS at 4° C. for 20 min. Samples were analyzed on a FACSCalibur flow cytometer (BD Biosciences). Results were analyzed using WinMDI software version 2.9 (Joseph Trotter).

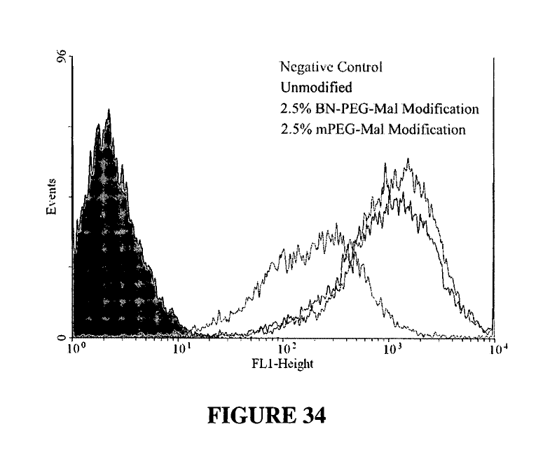

FIG. 34 shows the cellular uptake efficiency mediated by targeted nanoparticles (Blue, 2.5% BN-PEG-Mal modification) via receptor-mediated endocytosis was comparable to that of unmodified nanoparticles (Black) via non-specific endocytosis, yet significantly higher than that of 2.5% mPEG-Mal modified MFC/siRNA nanoparticles (Purple). The results suggest that pegylation of the nanoparticles decreased non-specific cellular uptake, and incorporation of targeting moiety resulted in enhancement of receptor-mediated endocytosis. Untreated cells were used as the negative control.

Cellular uptake of RGD-PEG-Mal modified Nanoparticles. Approximately 500,000 U87 cells were plated per well in a 6-well plate 24 hours prior to the study. Fluorescein-labeled MFC/siRNA nanoparticles were prepared as described above. After MFC/siRNA nanoparticles formation, appropriate amount of RGD-PEG-Mal or mPEG-Mal was added to the mixture. After 2 h incubation in dark, free maleimide derivatives were removed by ultrafiltration (Nanosep®, MWCO=100K, 5000 g, 5 min). The recovered samples were applied to cells and incubated at 37° C. After 2 h the medium was removed by aspiration and the cells were washed twice with cold phosphate buffered saline (PBS) and then trypsinized. The cells were collected and fixed with 2% PFA in PBS at 4° C. for 20 min. Samples were analyzed on a FACSCalibur flow cytometer (BD Biosciences).

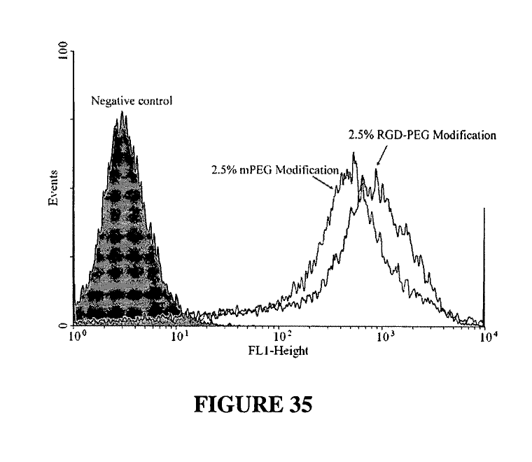

FIG. 35 shows the cellular uptake efficiency mediated by targeted nanoparticles (Blue, 2.5% RGD-PEG-Mal modification) was significant higher than that of non-targeted nanoparticles (Purple, 2.5% mPEG-Mal modification), suggesting that incorporation of RGD peptide in nanoparticles resulted in enhancement of receptor-mediated endocytosis. Untreated cells were used as the negative control.

Cellular uptake competition of RGD-PEG-Mal modified nanoparticles by pre-incubation of cyclo(RGDfK). Approximately 500,000 U87 cells were plated per well in a 6-well plate 24 hours prior to the study. Fluorescein-labeled RGD-PEG-Mal modified nanoparticles were prepared as described above. In competition experiments, the cells were pre-incubated with a 100-fold molar excess of cyclo(RGDfK) peptide for 30 min at 4° C. Subsequently, RGD-PEG-Mal modified nanoparticles were applied to cells and incubated at 37° C. After 2 h the medium was removed by aspiration and the cells were washed twice with cold phosphate buffered saline (PBS) and then trypsinized. The cells were collected and fixed with 2% PFA in PBS at 4° C. for 20 min. Samples were analyzed on a FACSCalibur flow cytometer (BD Biosciences).

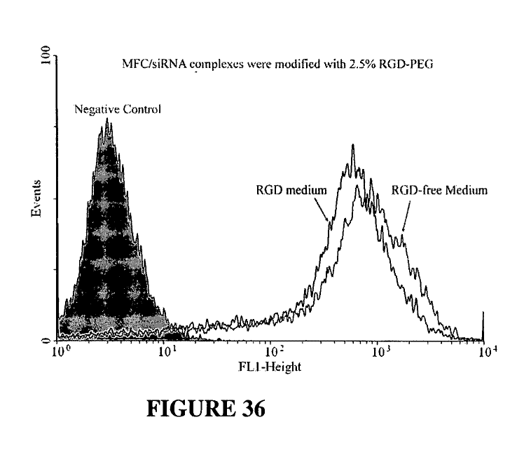

FIG. 36 shows the cellular uptake efficiency mediated by 2.5% RGD-PEG-Mal modified targeted nanoparticles. The cells which were pre-incubated with a 100-fold molar excess of cyclo(RGDfK) peptide exhibited lower uptake efficiency than that of the cells in RGD-free medium.

Intravenous Injection of Targeted MFC/siRNA Nanoparticles

Animal Tumor Model

U87 cells were collected and resuspensed with medium/matrigel mixture (v/v=1/1). 100 μL of cell suspension containing 2 million cells were injected subcutaneously in the right flanks of the mice. MFC-EHCO was used in the study. Hif is hypoxia-inducible factor-1a that is related to regulate the expression of VEGF, and silencing the expression of Hif by anti-Hif siRNA has been demonstrated to inhibit tumor growth. Anti-Hif siRNA was tested to inhibit tumor growth.

Administration of siRNA complexes and tumor size measurements Anti-Hif siRNA complexes were injected on day 21 after tumor cell inoculation. 200 μL of complexes containing 40 μg siRNA were injected into mice via the systemic i.v. administration. Each group of mice (n=5) were injected siRNA complexes through i.v. injection. In each group, mice were treated with nanoparticles or solution. Specifically, Group 1: PEI/siRNA complexes, Group 2: Naked siRNA, Group 3: mPEG modified EHCO/siRNA complexes (modification degree: 2.5%), Group 4: BN-PEG modified EHCO/siRNA complexes (modification degree: 2.5%), Group 5: RGD-PEG modified EHCO/siRNA complexes (modification degree: 2.5%). However, all of the mice treated with PEI/siRNA nanoparticles were dead right after injection, most likely due to high toxicity of PEI.

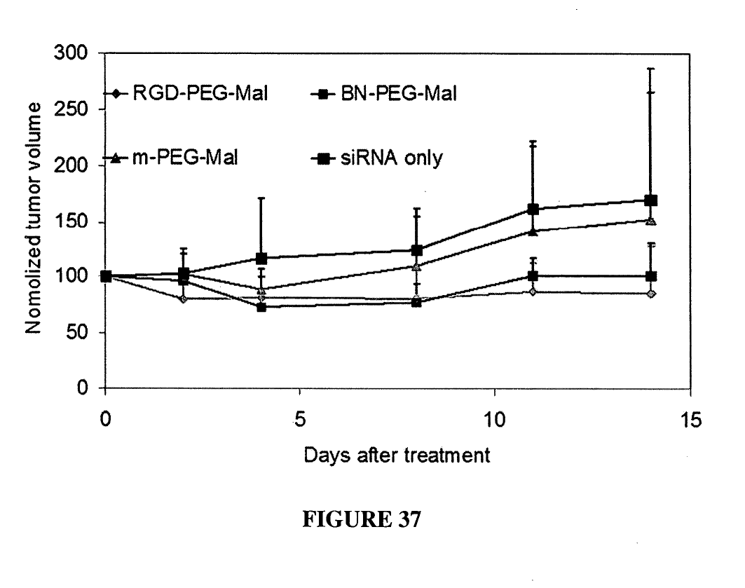

The growth of the tumors in the remaining mice was monitored every 2 to 4 days by measuring the tumor volume with a digital caliper. Tumor volumes were calculated using the formula: (⅙)πD₁²D₂, where D₁ is the smaller diameter measured. The day that mice received siRNA was set as day 0, and the tumor volume was normalized to 100%. All subsequent tumor volumes were expressed as the percentage relative to those at day 0. The peptide targeted siRNA nanoparticles inhibited tumor growth in two weeks after the treatment, while significant tumor growth was observed for the mice treated with non-targeted delivery system and free siRNA. FIG. 37 shows the i.v. administration of 2.5% peptide-modified EHCO/siRNA nanoparticles (BN-PEG-Mal, blue; or RGD-PEG-Mal, orange), where the nanoparticles significantly inhibited the mice tumor growth rate compared to non-targeting MFC/siRNA nanoparticles (2.5% mPEG-Mal modified nanoparticles) or naked siRNA solution.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

1. A compound comprising the formula I 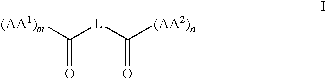
wherein AA¹ and AA² are each one or more amino acids, wherein (AA¹)ₘ and (AA²)ₙ are the same or different sequence;
m and n are an integer from 1 to 50; and
L comprises a group comprising at least one neutral amino group or cationic ammonium group, or the pharmaceutically-acceptable salt, ester, or amide thereof.

2. The compound of claim 1, wherein at least one amino acid of (AA¹)ₘ and (AA²)ₙ is a residue of cysteine, homocysteine or a thiol containing derivative of an amino acid.

3. The compound of claim 1, wherein a hydrophobic group is covalently attached to a least one amino acid of (AA¹)ₘ or (AA²)ₙ or structure L.

4. The compound of claim 1, wherein a targeting group is attached to a least one amino acid of (AA¹)ₘ or (AA²)ₙ or structure L.

5. The compound of claim 1, wherein the compound comprises the formula II 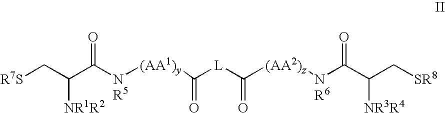
wherein R¹-R⁸ are, independently, hydrogen, an alkyl group, an alkenyl group, an acyl group, an aromatic group, or a hydrophobic group;
wherein AA¹ and AA² are one or more amino acids, wherein (AA¹)_{y} and (AA²)_{z} are the same or different sequence;
y and z are an integer from 0 to 50; and
L comprises a group comprising at least one neutral amino group or cationic ammonium group, or the pharmaceutically-acceptable salt, ester, or amide thereof.

6. The compound of claim 5, wherein L comprises the formula III 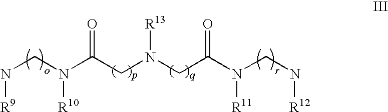
wherein R⁹-R¹² are, independently, hydrogen, an alkyl group, an alkenyl group, or an aromatic group;
R¹³ is an alkylamino group or a group containing at least one aromatic amino group; and
o, p, q, and r are, independently, an integer from 1 to 10.

7. The compound of claim 6, wherein the alkylamino group comprises the formula IV, V, or VI 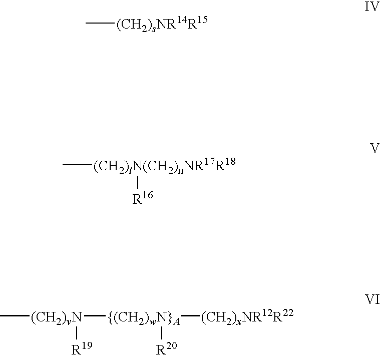
wherein R¹⁴-R¹²² are, independently, hydrogen, an alkyl group, a hydrophobic group; or a nitrogen containing substituent;
s, t, u, v, w, and x are an integer from 1 to 10; and
A is an integer from 1 to 50.

8. The compound of claim 6, wherein aromatic amino group comprises one or more amino groups directly or indirectly attached to the aromatic group or in the ring structure.

9. The compound of claim 6, wherein R¹-R¹² are hydrogen; o, p, q, an r are each 2; y and z are zero or one; and R¹³ is CH₂CH₂NHCH₂CH₂NHCH₂CH₂NH₂, CH₂CH₂NH₂, or CH₂CH₂CH₂NHCH₂CH₂CH₂CH₂NHCH₂CH₂CH₂NH₂.

10. The compound of claim 6, wherein R¹ and R³ are a hydrophobic group; R² and R⁴-R¹² are hydrogen; o, p, q, an r are each 2; y and z are zero or one; and R¹³ is CH₂CH₂NHCH₂CH₂NHCH₂CH₂NH₂, CH₂CH₂NH₂, or CH₂CH₂CH₂NHCH₂CH₂CH₂CH₂NHCH₂CH₂CH₂NH₂.

11. The compound of claim 1, wherein the compound is EHCO, SHCO, or THCO.

12. A compound comprising the formula VII 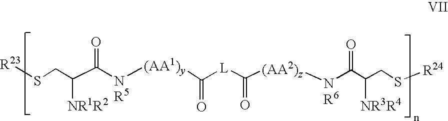
R¹-R⁶ are, independently, hydrogen, an alkyl group, an alkenyl group, an aromatic group, or a hydrophobic group;
AA¹ and AA² are one or more amino acids, wherein (AA¹)_{y} and (AA²)_{z} are the same or different sequence;
y and z are an integer from 0 to 50;
R²³ and R²⁴ are, independently, hydrogen or a targeting group;
B is an integer from 2 to 10,000; and
L comprises a group comprising at least one neutral amino group or cationic ammonium group, or the pharmaceutically-acceptable salt, ester, or amide thereof.

13. The compound of claim 12, wherein L comprises from 1 to 50 protonatable amino or amine groups.

14. The compound of claim 12, wherein L comprises from 1 to 50 protonatable amino or amine groups.

15. The compound of claim 12, wherein polyethylene glycol is covalently attached to the compound.

16. The compound of claim 12, wherein a targeting group is covalently attached to the compound by a linker.

17. The compound of claim 16, wherein the linker comprises a polyamino acid group, a polyalkylene group, or a polyethylene glycol group.

18. The compound of claim 16, wherein the targeting group comprises a peptide, a protein, an antibody, an antibody fragment or one of their derivatives.

19. The compound of claim 16, wherein the targeting group comprises an RGD peptide or bombesin peptide.

20. The compound of claim 12, wherein at least one amino acid comprises histidine.

21. A disulfide oligomer or polymer produced by the process comprising reacting the same or different compound of claim 1 in the presence of an oxidant.

22. A nanosized complex comprising a nucleic acid and one or more compound carriers 1-21 of claim 1.

23. The complex of claim 22, wherein the complex further comprises a targeting agent.

24. The complex of claim 23, wherein the targeting agent comprises a peptide, a protein, an antibody, an antibody fragment or one of their derivatives.

25. The complex of claim 23, wherein the targeting agent comprises an RGD peptide or bombesin peptide.

26. The complex of claim 23, wherein the targeting agent is covalently attached to the nanosized complex via a linker.

27. The complex of claim 26, wherein the linker comprises a polyamino acid group, a polyalkylene group, or a polyethylene glycol group.

28. The complex of claim 22, wherein the nucleic acid comprises, a natural or synthetic oligonucleotide, a natural or modifiediblocked nucleotide/nucleoside, a DNA or fragment there of, or an RNA or fragment there of.

29. The complex of claim 22, wherein the nucleic acid comprises a siRNA.

30. The complex of claim 22, wherein the nucleic acid comprises a plasmid DNA.

31. A complex produced by the process comprising admixing a nucleic acid and one or more compound carriers of claim 1.

32. The complex of claim 1, wherein the complex undergoes cellular membrane disruption at a pH of 5 to 6.

33. The complex of claim 1, wherein the complex undergoes cellular membrane disruption at a pH of 5 to 5.5.

34. A method for introducing a nucleic acid into a cell comprising contacting the cell with a complex of claim 1, wherein the nucleic acid is taken up into the cell.

35. The method of claim 34, wherein said contacting step occurs in vitro.

36. The method of claim 34, wherein said contacting step occurs in vivo.
